(19) United States
(12) Patent Application Publication
(10) Pub. No.: US 2017/0296485 A1
(43) Pub. Date: Oct. 19, 2017

(54) Topical Film-Forming Spray

(71) Applicant: GRACE THERAPEUTICS LLC, New Brunswick, NJ (US)

(72) Inventors: S. George Kottayil, West Windsor, NJ (US); Amresh Kumar, Plainsboro, NJ (US); Prasanna Sunthankar, West Windsor, NJ (US); Vimal Kavuru, Holmdel, NJ (US)

(73) Assignee: GRACE THERAPEUTICS LLC, New Brunswick, NJ (US)

(21) Appl. No.: 15/360,676

(22) Filed: Nov. 23, 2016

Related U.S. Application Data
(60) Provisional application No. 62/258,672, filed on Nov. 23, 2015.

(51) Int. Cl. A61K 9/70; A61K 9/00; A61K 31/445; A61K 45/06

(52) U.S. Cl. CPC A61K 9/7015; A61K 45/06; A61K 9/0014; A61K 31/445

(57) ABSTRACT

A polymeric bio-adhesive film forming topical spray formulation providing a modified, pulsatile (e.g., biphasic) release of the active agent(s) once the solvent evaporates and the film sets, e.g., on human skin is disclosed. In certain embodiments, the active agent is bupivacaine hydrochloride.

This application claims priority from U.S. Provisional Application No. 62/258,672, filed Nov. 23, 2015, the disclosure of which is hereby incorporated by reference in its entirety.

### FIELD OF THE INVENTION

The present invention relates to a film-forming composition which forms a stable film when applied (e.g., sprayed) on a surface (e.g., skin). The stable film is formed by evaporation of solvent and adheres to the surface.

### BACKGROUND OF THE INVENTION

Topical films that incorporate therapeutic agents have been developed for a variety of purposes in the pharmaceutical arts. For example, Tipton et al. (U.S. Pat. Nos. 5,632,727 and 5,792,469) describes the preparation of a biodegradable film dressing with or without additional therapeutic agents formed from a liquid composition of at least one biodegradable/bioerodible thermoplastic polymer in a pharmaceutically acceptable solvent. The film is formed by dispensing, preferably by spraying, the liquid composition onto a tissue site and contacting the liquid composition with an aqueous-based fluid to coagulate or solidify the film onto the human or animal tissue. The biodegradable film can be used to protect and to promote healing of injured tissue and/or to deliver biologically active agents.

U.S. Pat. No. 6,958,154 describes a spray-on bandage and drug delivery system. Therein, a fluid composition, e.g., an aerosol spray, is applied onto a surface as a fluid, but then dries in situ to form a patch having a tack-free outer surface covering an underlying adhesive that helps to adhere the patch to the substrate.

U.S. Pat. No. 6,899,897 describes the use of a natural gum resin as a carrier for topical application of an active agent. The biological dressing described therein is comprised of a gum resin, a topically acceptable volatile solvent, and a pharmacologically active agent. The gum resin is present in a suitable amount that the composition, when the solvent evaporates, will dry to form a solid coating that sticks to the skin or mucosal membrane to which the composition is applied and maintain the pharmacologically active agent over a sustained period of time in contact with sites on the skin or mucosal membranes exhibiting symptoms of a disease to be treated.

However, conventional topical spray formulations tend to remain at the application site for only a short time and as a result, the active agent is absorbed through the skin is only available transiently. Further, known topical spray formulations may cause skin irritation or occlusion problems.

### OBJECTS AND SUMMARY OF THE INVENTION

It is an object of the present invention to provide formulations and methods for providing pain relief in humans and animals by administering a dose of a local anesthetic formulation to an open wound or surgical site for the treatment of acute or chronic pain, nociceptive and neuropathic pain.

It is an object of the present invention to provide formulations and methods for providing pain relief in humans and animals by administering a dose of a local anesthetic formulation for the treatment of pre- and post-operative pain, cancer pain, pain associated with neurotransmitter dysregulation syndromes and orthopedic disorders, and/or localized severe or intractable pain.

The present invention in certain embodiments is directed to a method of treating pain comprising the administration of a polymeric film forming spray formulation which is applied to the skin via the utilization of a (mechanical pump) spray device.

In certain embodiments, the invention is directed in part to a polymeric film forming topical spray formulation, comprising a hydrophilic film forming polymer, the hydrophilic film forming polymer being present in the formulation in an amount from about 1% to about 50%, or about 2% to about 50% by weight, a drug crystal precipitation inhibiting agent in an amount effective to prevent or substantially prevent the active agent(s) included in the formulation from precipitating a pharmaceutically acceptable permeation enhancer for the active agent(s) and wherein the hydrophilic film forming polymer and the drug crystal precipitation inhibiting agent are the same or different, an active agent(s), a pharmaceutically acceptable permeation enhancer for the active agent(s), and a volatile solvent in a concentration from about 20 to about 99% of the formulation, by weight, the formulation when sprayed on and set on human skin provides a breathable, bioadhesive and microporous film releasing from about 0.001% to about 25% of the active agent within about 2 hours, and further providing a biphasic release of the active agent(s). Preferably, the topical spray formulation contains a therapeutically effective amount (or concentration) of the active agent(s). The invention is further directed to a polymeric film forming topical spray formulation, comprising a hydrophilic film forming polymer, the hydrophilic film forming polymer being present in the formulation in an amount from about 2 to about 50%, by weight, an active agent, a drug crystal precipitation inhibiting agent in an amount effective to prevent or substantially prevent the active agent included in the formulation from precipitating, a pharmaceutically acceptable permeation enhancer for the active agent, wherein the hydrophilic film forming polymer and the drug crystal precipitation inhibiting agent are the same or different, and a volatile solvent in a concentration from about 20 to about 99% of the formulation, by weight, the formulation when sprayed on and set on a site on human skin providing a breathable, bioadhesive and microporous film and further providing a biphasic release of the active agent(s) such that the formulation provides a first peak concentration of the active agent at about 0.05 to about 5 hours after application of the topical spray on the skin of a human subject, and provides a second peak concentration of the active agent at about 3 hours to about 24 hours after application of the topical spray on the skin of a human subject. In certain preferred embodiments, the topical spray formulation provides an initial release of active agent(s) once the topical spray is sprayed onto human skin followed by a lag during which less drug is released over a period of time. In certain preferred embodiments, the topical spray formulation provides a biphasic release of the active agent(s) in which a first portion of the active agent(s) is released either immediately or after a short time delay to provide a first peak maximum concentration at the site (for a topical drug) or in blood plasma (for a systemic treatment/drug) which occurs from about 0.05 to about 5 hours or from about 0.5 hours to about 5 hours after application of the topical spray on the skin, and a second portion of the active agent(s) released after a lag time to provide a second peak maximum concentration from about 3 hours to about 24 hours or from about 3 hours to about 15 hours after application of the topical spray on the skin. In certain preferred embodiments, the formulation provides a cumulative drug permeation in-vitro from about 10 μg/cm² to about 6500 μg/cm², performed on Mattek Epiderm or cadaver skin using In-line PermeGear ILC07 automatic diffusion cell system. In certain preferred embodiments, the formulation comprises a supersaturated concentration of the drug (e.g., bupivacaine). This may result in the drug concentration lasting for an extended period of time in vivo (e.g., from about 1 to about 24 hours, or from about 3 to about 12 hours) and may provide increased bioavailability of the drug from the formulation. In certain preferred embodiments, the topical polymeric film forming composition provides an in-vitro cumulative drug permeation from about 10 μg/cm² to about 500 μg/cm² after 2 hours, and a cumulative in-vitro drug permeation from 10 μg/cm² to 6500 μg/cm² after 24 hours. In other preferred embodiments, the topical polymeric provides an in-vivo cumulative drug permeation on human skin from about 10 ng/cm² to about 500 ng/cm² after 2 hours, and a cumulative drug permeation in-vivo from 10 ng/cm² to 6500 ng/cm² after 24 hours. In certain preferred embodiments, the drug crystal precipitation inhibiting agent comprises from about 0.01 to about 50%, preferably from about 2 to about 20%, more preferably from about 2.5 to about 10% of the formulation. The topical spray formulation preferably provides a biphasic release of the active agent(s) in which a first portion of the active agent(s) is released either immediately or after a short time delay to provide a first peak maximum concentration (i) at the site when the drug is topically active), or (ii) in blood plasma when the drug is systemically active which occurs from about 0.05 hours to about 5 hours after application of the topical spray on the skin, and a second portion of the active agent(s) released after a lag time to provide a second peak maximum concentration from about 3 hours to about 24 hours after application of the topical spray on the skin.

In certain embodiments, the formulation provides a cumulative in-vitro drug (e.g., bupivacaine) permeation from about 10 μg/cm² to about 6500 μg/cm², performed for example on Mattek Epiderm or cadaver skin using In-line PermeGear ILC07 automatic diffusion cell system.

The present invention is further directed in part to a pharmaceutical formulation comprising a polymeric solution, emulsion or suspension of a hydrophilic polymer, an active agent(s), and a pharmaceutically acceptable permeation enhancer dispersed in a pharmaceutically acceptable hydroalcoholic solvent, the formulation capable of being sprayed via a pump spray into droplets having a diameter from about 5 microns to about 1000 microns and setting as a microporous, breathable and bioadhesive film when the hydroalcoholic solvent evaporates, and releasing from about 0.001% to about 25% of the active agent within about 2 hours, and further providing a biphasic release of the active agent(s) when the formulation sets as a film on human skin.

The present invention in certain embodiments is directed to a topical polymeric film forming composition, comprising bupivacaine hydrochloride dispersed in a hydroalcoholic solvent together with a hydrophilic film forming polymer in an amount from about 2 to about 50%, by weight a drug crystal precipitation inhibiting agent in an amount effective to prevents or substantially prevent the drug(s) included in the formulation from precipitating a pharmaceutically acceptable permeation enhancer for the active agent(s) and wherein the hydrophilic film forming polymer and the drug crystal precipitation inhibiting agent are the same or different, and a permeation enhancer, the formulation being capable of being sprayed as a unit dose onto skin via the use of a pump spray to provide droplets having a diameter from about 1 to about 1000 microns, when sprayed and set on skin providing a breathable, bioadhesive and microporous film. In certain preferred embodiments, the formulation includes one or more drug crystal precipitation inhibiting agent in an amount from about 0.01 to about 50%, or from about 2 to about 20% of the formulation, or from about 2.5 to about 10% of the topical spray formulation. The drug crystal precipitation inhibiting agent may be the same or different than the hydrophilic film forming polymer. In certain preferred embodiments, the topical polymeric film forming composition, which when sprayed and set on human skin (e.g., to an affected area of the human) releases from about 0.001% to about 25% of the bupivacaine within about 2 hours, and further providing a biphasic release of the bupivacaine. In certain preferred embodiments, the topical polymeric film forming composition provides an in-vitro cumulative drug permeation from about 10 μg/cm² to about 500 μg/cm² after 2 hours, and a cumulative in-vitro drug permeation from 10 μg/cm² to 6500 μg/cm² after 24 hours. In other preferred embodiments, the topical polymeric provides an in-vivo cumulative drug permeation on human skin from about 10 ng/cm² to about 500 ng/cm² after 2 hours, and a cumulative drug permeation in-vivo from 10 ng/cm² to 6500 ng/cm² after 24 hours. In certain preferred embodiments, the topical polymeric film forming composition provides a biphasic release profile in-vivo. The topical spray formulation provides a biphasic release of the active agent(s) in which a first portion of the active agent(s) is released either immediately or after a short time delay to provide a first peak maximum concentration (i) at the site when the drug is topically active), or (ii) in blood plasma when the drug is systemically active which occurs from about 0.05 hours to about 5 hours after application of the topical spray on the skin, and a second portion of the active agent(s) released after a lag time to provide a second peak maximum concentration from about 3 hours to about 24 hours after application of the topical spray on the skin. In preferred embodiments, the topical polymeric film forming composition provides a biphasic release when sprayed and set on human skin and having a first and second phase of release, wherein the first phase of drug release reaches a peak at from about 0.5 to about 3 hours and the second phase of release reaches a peak plasma concentration at about 3 to about 15 hours or at about 3 to about 15 hours or about 3 to about 7 hours after application of the unit dose on human skin. In other preferred embodiments, the biphasic release provides a first and second phase of release, wherein the first phase of bupivacaine release reaches a peak at from about 0.25 to about 1.5 hours and the second phase of bupivacaine release reaches a peak plasma concentration at from about 4 to about 12 hours after application of the unit dose on human skin. In certain embodiments, the formulation provides a bupivacaine dose from about 0.5 to about 40 mg, or from about 1 mg to about 20 mg bupivacaine, sprayed as a unit dose onto the skin, to provide a film surface area from about 1 cm² to about 40 cm² per spray. The unit dose may be comprised, e.g., of from 1 to about 20 sprays per application of the unit dose onto the skin.

The invention is also directed to a unit dose of a topical spray pharmaceutical formulation comprising a polymeric solution, emulsion or suspension of a hydrophilic polymer, a drug crystal precipitation inhibiting agent, an active agent, and a pharmaceutically acceptable permeation enhancer dispersed in a pharmaceutically acceptable hydroalcoholic solvent, the unit dose comprising a plurality of spray droplets, wherein 10% of the spray droplets in the unit dose have a mean diameter of about 26 μm±20 μm, about 50% of the spray droplets in the unit dose have a mean diameter of about 55 μm±20 μm, and about 90% of the spray droplets in the unit dose have a mean diameter of about 116 μm±40 μm, the unit dose topical spray provides a film surface area from about 1 cm² to about 40 cm² per spray and sets as a microporous, breathable and bioadhesive film when the hydroalcoholic solvent evaporates and provides a biphasic release of the active agent. In certain embodiments, a dose of the active agent is sprayed as a unit dose onto the skin, to provide a film surface area, e.g., from about 1 cm² to about 40 cm², and in certain embodiments preferably about 20 cm² per spray. The unit dose may be administered by spraying from about 1 to about 20 sprays (which would be, e.g., 20×20 cm²) or from 1 to about 7 sprays per application of the unit dose onto the skin. In certain preferred embodiments, the hydrophilic film forming polymer and drug crystal precipitation inhibiting agent comprise povidone. Preferably, the hydrophilic polymer comprises from about 2 to about 50% of the formulation, and the drug crystal precipitation inhibiting agent comprises from about 0.01 to about 50% of the formulation, and more preferably from about 2.5 to about 10% of the formulation, by weight. In certain preferred embodiments, the biphasic release provides a first peak concentration of the active agent at from about 0.5 to about 3 hours and the second peak concentration of the active agent at from about 3 to about 15 hours after application of the unit dose on human skin. In certain embodiments, the biphasic release provides a first peak at from about 0.5 to about 3 hours and a second peak at from about 3 to about 7 hours after application of the unit dose on human skin. In certain preferred embodiments, 10% of the spray droplets in the unit dose have a mean diameter of about 26 μm±1.82 μm, about 50% of the spray droplets in the unit dose have a mean diameter of about 55 μm±2.39 μm, and about 90% of the spray droplets in the unit dose have a mean diameter of about 116 μm±4.9 μm.

In certain embodiments, the active agent comprises from about 0.5 to about 40 mg bupivacaine hydrochloride. In certain preferred embodiments, the bupivacaine concentration in the formulation is supersaturated. The supersaturated drug concentration may last for a time period from about 1 to about 24 hours in vivo, to achieve increased bioavailability. In certain preferred embodiments, the first peak concentration of bupivacaine occurs at from about 0.17 to about 0.67 hours after the unit dose is sprayed onto the human subject, and the second peak concentration of bupivacaine occurs at from about 4 to about 24 hours after the unit dose is sprayed onto the human subject. In certain embodiments, the unit dose provides a first peak plasma concentration from about 29 pg/ml to about 380 pg/ml bupivacaine, and a second peak plasma concentration from about 864 pg/ml to about 3463 pg/ml bupivacaine. The unit dose containing the bupivacaine may be, e.g., 12 mg, and the dose may be administered as 12 mg per spray.

In certain embodiments, the invention is directed to a method of treating neuropathic pain, comprising spraying a unit dose of a topical polymeric film forming composition comprising bupivacaine hydrochloride dispersed in a hydroalcoholic solvent together with a hydrophilic film forming polymer in an amount from about 2 to about 50%, by weight and a permeation enhancer onto the skin of a human patient, such that the unit dose is sprayed onto the skin in the form of droplets having a diameter from about 1 to about 1000 microns and the droplets, when set on the skin providing a breathable, bioadhesive and microporous film which provides a biphasic release of the bupivacaine. In certain embodiments, a bupivacaine dose from about 1 to about 20 mg is sprayed as a unit dose onto the skin, to provide a film surface area from about 1 cm² to about 40 cm², preferably about 20 cm² per spray, further comprising spraying from about 1 to about 20 sprays or from 1 to about 7 sprays per application of the unit dose onto the skin. In certain preferred embodiments, the method further comprises spraying a further unit dose of the topical polymeric film forming composition onto the skin of the human patient about 6 hours after application of a first unit dose. In certain preferred embodiments, the unit dose is sprayed using a metered pump delivering from about 40 μl to about 350 μl volume per spray. In certain embodiments, the biphasic release provides a first and second phase of release, wherein the first phase of bupivacaine release reaches a peak at about 0.5 to about 3 hours and the second phase of bupivacaine release reaches a peak plasma concentration at about 3 to about 15 hours or at about 3 to about 7 hours after application of the unit dose on human skin.

In certain embodiments, the bupivaicaine topical spray droplet size from about 20 to about 31 μm Dv (10), and/or a spray droplet size from about 49 to about 60 μm Dv (50), and/or a topical spray droplet size from about 100 to about 140 μm Dv (90).

The invention is further directed in part to a method of treating neuropathic pain (e.g., erythromelalgia, post-herpetic neuralgia, fibromyalgia and/or complex regional pain syndrome (CRPS), among others) comprising spraying a unit dose of a topical polymeric film forming composition comprising a local anesthetic (e.g., bupivacaine base, bupivacaine hydrochloride or a mixture thereof) dispersed in a hydroalcoholic solvent together with a hydrophilic film forming polymer in an amount from about 2 to about 50%, by weight an effective amount of a drug crystal precipitation inhibiting agent, and a permeation enhancer onto the skin of a human patient, such that the unit dose containing a therapeutically effective amount of the local anesthetic is sprayed onto the skin in the form of droplets having a diameter from about 1 to about 1000 microns and the droplets, when set on the skin providing a breathable, bioadhesive and microporous film which provides a biphasic release of the bupivacaine. The bupivacaine dose may be from about 0.5 to about 40 mg is sprayed as a unit dose onto the skin, to provide a film surface area from about 1 cm² to about 40 cm² per spray, and may be dosed by spraying from 1 to about 20 sprays per application of the unit dose onto the skin. In certain embodiments, a further unit dose of the topical polymeric film forming composition may be sprayed onto the skin of the human patient about 6 hours after application of a first unit dose. The unit dose may be sprayed using a metered pump delivering, e.g., from about 40 μl to about 350 μl volume per spray. The biphasic release preferably provides a first and second phase of release, wherein the first phase of bupivacaine release reaches a peak at from about 0.25 to about 1.5 hours and the second phase of bupivacaine release reaches a peak plasma concentration at from about 4 to about 12 hours after application of the unit dose on human skin.

The invention is also directed in part to a method of treating pain, comprising spraying onto the skin of a human a unit dose of a topical spray pharmaceutical formulation comprising a polymeric solution, emulsion or suspension of a hydrophilic polymer, a drug crystal precipitation inhibiting agent, an effective amount of an active agent suitable for treating pain, and a pharmaceutically acceptable permeation enhancer dispersed in a pharmaceutically acceptable hydroalcoholic solvent, the unit dose comprising a plurality of spray droplets, wherein 10% of the spray droplets in the unit dose have a mean diameter of about 26 μm±20 μm, about 50% of the spray droplets in the unit dose have a mean diameter of about 55 μm±20 μm, and about 90% of the spray droplets in the unit dose have a mean diameter of about 116 μm±40 μm, such that the unit dose topical spray provides a film surface area from about 1 cm² to about 40 cm² per spray and sets as a microporous, breathable and bioadhesive film when the hydroalcoholic solvent evaporates and provides a biphasic release of the active agent.

The invention is further directed in part to a method of treating pain, comprising spraying onto the skin of a human in proximity to an affected area a unit dose of a topical spray pharmaceutical formulation comprising a polymeric solution, emulsion or suspension of a hydrophilic polymer, a drug crystal precipitation inhibiting agent, an effective amount of an active agent comprising a local anesthetic or a pharmaceutically acceptable salt thereof, and a pharmaceutically acceptable permeation enhancer dispersed in a pharmaceutically acceptable hydroalcoholic solvent, the unit dose comprising a plurality of spray droplets, wherein 10% of the spray droplets in the unit dose have a mean diameter of about 26 μm±20 μm, about 50% of the spray droplets in the unit dose have a mean diameter of about 55 μm±20 μm, and about 90% of the spray droplets in the unit dose have a mean diameter of about 116 μm±40 μm, such that the unit dose topical spray sets as a microporous, breathable and bioadhesive film having a film surface area from about 1 cm² to about 40 cm² per spray and when the hydroalcoholic solvent evaporates and provides a biphasic release of the active agent.

In certain embodiments, the methods further comprise using a metered pump delivering, e.g., from about 40 μl to about 350 μl volume per spray to deliver the active agent onto the skin of the human.

For purposes of the present invention, the terms “active agent”, “drug” and medicament are used interchangeably, and are meant to encompass a single drug or multiple drugs (two or more) contained in the topical spray formulations of the invention.

The term “local anesthetic” means any drug or mixture of drugs that provides local numbness and/or analgesia.

As used herein, the term “unit dose” refers to physically discrete units suitable as unitary dosages for mammalian subjects, each unit containing as the active ingredient a predetermined quantity of the active agent (e.g., local anesthetic).

The term “comprising” is an inclusive term interpreted to mean containing, embracing, covering or including the elements listed following the term, but not excluding other unrecited elements.

For purposes of the invention, the term “controlled release”, “sustained release”, and similar terms are used to denote a mode of active agent delivery that occurs when the active agent is released from the delivery vehicle at an ascertainable and controllable rate over a period of time, rather than dispersed immediately upon application or injection. The controlled or sustained release formulations of the invention preferably provide a sustained action in the localized area to be treated. For example, it would be desirable that such a formulation provides localized anesthesia to the site for a period of, e.g., one day. The formulations can therefore, of course, be modified in order to obtain such a desired result.

The term “modified release” as used herein in relation to the composition according to the invention or a coating or coating material or used in any other context means release which is not immediate release and is taken to encompass controlled release, sustained release and delayed release.

A “therapeutically effective amount” means the amount that, when administered to an animal for treating a disease, is sufficient to effect treatment for that disease.

As used herein, the term “treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). For the purposes of this invention, a “disease” includes pain.

As used herein, the term “bioerodible” refers to the degradation, disassembly or digestion of the sustained release carrier of the gel formulation by action of a biological environment, including the action of living organisms and most notably at physiological pH and temperature.

As used herein, the term “topically active” refers to a film formulation in accordance with the present invention which includes at least one active agent which treats predominately the surface on which it is applied.

As used herein, the term “transdermal” refers to a film formulation in accordance with the present invention which includes at least one active agent that is absorbed through skin when applied topically. Typically, although not necessarily, the medicament will then be distributed throughout the body resulting in systemic action as opposed to being only locally active at the site of application.

For purposes of the present invention, the term “active agent” is meant to encompass, but not be limited to, a drug. The term active agent is further meant to encompass a single active agent, or multiple (two or more) active agents present in the formulation.

For purposes of the present invention, all percentages described herein are “w/w” unless otherwise specified.

### BRIEF DESCRIPTION OF THE DRAWINGS

FIG. 1 depicts an in-vivo drug plasma concentration vs time plot of individual rats treated with a bupivacaine (HCl and base) metered dose transdermal film sprays in accordance with the present invention.

FIG. 2 depicts an in-vivo drug plasma concentration vs time plot of individual rats treated with a bupivacaine metered dose transdermal film spray in accordance with the present invention.

FIG. 3 depicts an in-vivo average drug plasma concentration vs time plot of 8 mg, 12 mg and placebo of individual rats treated with a bupivacaine metered dose transdermal film spray in accordance with the present invention.

FIG. 4 depicts an in-vivo average cumulative drug plasma concentration vs time plot of 8 mg, 12 mg and placebo of individual rats treated with a bupivacaine metered dose transdermal film spray in accordance with the present invention.

FIG. 5A depicts bupivacaine topical spray film characteristics of a bupivacaine spray formulation according to the present invention without PVP.

FIG. 5B depicts bupivacaine topical spray film characteristics of a bupivacaine spray formulation according to the present invention with 0.5% PVP.

FIG. 5C depicts bupivacaine topical spray film characteristics of a bupivacaine spray formulation according to the present invention with 2.5% PVP.

FIG. 5D depicts bupivacaine topical spray film characteristics of a bupivacaine spray formulation according to the present invention with 5% PVP.

FIG. 5E depicts bupivacaine topical spray film characteristics of a bupivacaine spray formulation according to the present invention with 10% PVP.

FIG. 5F depicts bupivacaine topical spray film characteristics of a bupivacaine and ethanol spray formulation.

FIG. 6 depicts the spray pattern and film uniformity of bupivacaine spray formulations according to the present invention containing different concentrations of PVP.

FIG. 7 is a graph depicting the bupivacaine plasma concentration (pg/mL) over 216 hours for the bupivacaine topical spray of Example 42.

FIG. 8 is a graph depicting the bupivacaine plasma concentration over 24 hours for the bupivacaine topical spray of Example 42.

### DETAILED DESCRIPTION

The invention will be described with reference to various specific and preferred embodiments and techniques, however, it should be understood that many variations and modifications can be made while remaining with the spirit and scope of the invention.

The polymeric film forming preparations produce a stable film in situ after application on the skin or any other body surface. These compositions can either be liquids or semisolids with a hydrophobic and or hydrophilic film forming polymer as basic material for the matrix. The formed film is sufficiently stable to provide a drug release to the skin from 1 hour up to 24 hours. The polymeric solution is sprayed to the skin as a liquid and forms an almost invisible film in situ by volatile solvent evaporation. The film is peal-able and/or non-pealable in nature. Preferably, the film is microporous, nanoporous, or both. The film is preferably breathable, meaning that it does not interfere with perspiration, respiration and other metabolic activities of the skin. The film is preferably non-greasy and non-sticky. It is also preferably non-transferable and is preferably bioadhesive.

The present invention is directed in certain embodiments to a film forming topical spray composition containing an active agent (such as bupivacaine or a pharmaceutically acceptable salt thereof). The composition in certain embodiments comprises the active agent, a film forming polymer, a volatile solvent, and a permeation enhancer (e.g., propylene glycol, oleyl alcohol, polyethylene glycol ethers of oleyl alcohol (e.g., Oleth-2), combinations thereof, and the like). In certain embodiments, the composition is dispensed via a metered dose device.

In preferred embodiments, the polymeric film forming spray formulations of the invention provide a modified release of the active agent(s) when applied or sprayed onto an environment of use (e.g., human skin). In preferred embodiments, the permeation or release of the drug following topical application from the invention is characterized by a biphasic release profile, the first phase comprising an immediate release of drug from the drug-solvent micro-droplet that is facilitated by the small (e.g., approximately 50 microns) spray droplet size and a subsequent second controlled release phase comprising release of drug from the bio-adhesive film formed, upon evaporation of the solvent system.

In preferred embodiments, the polymeric film forming topical spray formulation provides a modified, pulsatile release of the active agent(s) once the solvent evaporates and the film sets, e.g., on human skin. In such embodiments, modified release composition capable of releasing the active ingredient in a bimodal or multi-modal manner in which a first portion of the active ingredient is released either immediately or after a short time delay (preferably less than 1.5 hours) to provide a pulse of drug release and one or more additional portions of the active ingredient are released each after a respective lag time to provide at least one additional pulse of drug release.

The polymeric film forming topical spray formulation of the invention, by virtue of and dependent upon the choice and amount of hydrophilic polymer and the use of a hydroalcoholic solvent, has been found to provide an initial release of drug once the topical spray is applied (e.g., sprayed) onto a surface (e.g., human skin) followed by a lag during which less drug is released over a period of time. The duration of the lag time may be varied by altering the polymer composition and/or the amount of polymer and/or the choice of permeation enhancer and the form of the active agent (e.g., base or salt form, and choice of salt).

In certain preferred embodiments, the polymeric film forming topical spray formulation provides a biphasic release of the active agent(s) in which a first portion of the active agent(s) is released either immediately or after a short time delay to provide a first peak maximum concentration at the site (for a topical drug) or in blood plasma (for a systemic treatment/drug) which occurs from about 0.05 hours to about 5 hours after application of the topical spray on the skin, and a second portion of the active agent(s) released after a lag time to provide a second peak maximum concentration at about 3 hours to about 24 hours or at about 3 hours to about 15 hours after application of the topical spray on the skin. In certain embodiments, the first peak maximum concentration at the site (for a topical drug) or in blood plasma (for a systemic treatment/drug) occurs from about 0.05 or about 0.25 hours to about 1.5 hours after application of the topical spray on the skin, and a second portion of the active agent(s) released after a lag time to provide a second peak maximum concentration from about 4 hours to about 12 or to about 15 hours after application of the topical spray on the skin.

In preferred embodiments of the invention, the topical spray formulation provides a unit dose (e.g., comprising from about 1 to about 20 sprays) wherein 10% of the spray droplets in the unit dose have a mean diameter of about 26 μm±20 μm, about 50% of the spray droplets in the unit dose have a mean diameter of about 55 μm±20 μm, and about 90% of the spray droplets in the unit dose have a mean diameter of about 116 μm±40 μm. It is believed that the smallest spray droplets provide the initial quick (first) peak concentration and the drug may even be absorbed/released at the site of application prior to the setting of the film. Thereafter, the film sets and there is a delay until the second peak concentration occurs.

In certain preferred embodiments, the drug is supersaturated in the formulation. It is believed that the closer to supersaturation the drug is in the formulation, the more permeation is obtained when the drug is administered, e.g., by spraying onto human skin.

In a preferred embodiment of the present invention, the active ingredient is a local anesthetic in base or salt form, e.g., bupivacaine hydrochloride, and the composition in operation delivers the active ingredient in a bimodal or pulsed manner.

In another preferred embodiment of the present invention, the active ingredient is methylphenidate and the composition in operation delivers the active ingredient in a bimodal or pulsed manner. Such a composition in operation produces a plasma profile which substantially mimics that obtained by the sequential administration of two IR doses as, for instance, in a typical methylphenidate treatment regime.

In present invention micro-emulsion, nano-emulsion and/or solid lipid nanoparticles can be suspended and or dispersed in the polymeric film forming solution for dermal spray delivery of pharmaceutical active ingredients. Micro-emulsions are system well known for their long-term stability and ease of preparation. They are stabilized by an interfacial film of surfactants, usually in combination with co-surfactants such as short chain alcohols. Micro-emulsions offer several advantages for improving local delivery and efficiency of drugs through a high solubilization capacity, increased thermodynamic activity and a reduction of the diffusional barrier of the stratum corneum. In the present invention, micro-emulsion and nano-emulsion systems are used to increase the permeability of drug (e.g., bupivacaine salts and base) through the skin after spray administration by a mechanical pump.

The polymeric solution containing an active agent(s) is preferably sprayed (e.g. via a mechanical sprayer) as microdroplets and nanodroplets onto the skin of a mammal (e.g., a human subject or a human patient). In certain preferred embodiments, a unit dose of the active agent(s) is sprayed using a metered pump delivering from about 40 ul to about 350 ul volume per spray. Once the solvent evaporates, the film sets as a non-contiguous, highly nano- or micro-porous film. Each micro- and nano-droplet may be considered to act as a separate drug delivery system.

The polymeric film forming spray formulation can be used with any pharmaceutical active ingredient in the salt or base form or in a combination of two or more active ingredients that is stable on mixing with polymer and other excipients for effective topical drug delivery.

The present invention is a multi-dose and or unit dose of a topical spray formulation, this formulation comprising liquid droplets of an active agent(s) and a pharmaceutically acceptable solvent carrier. The droplets preferably have a size distribution of from about 1 micron to about 1000 microns, and in certain preferred embodiments from about 5 microns to about 500 microns in diameter. In certain preferred embodiments, the film does not set (dry) on the applied surface (e.g., human skin) as a contiguous sheet, but instead sets as a breathable, microporous (and nanoporous) film. The drug(s) is absorbed slowly at the site of application as the drug leaches into the skin from the film. It is hypothesized that each microdroplet/nanodroplet of the topical spray acts as a separate drug delivery system on the skin.

In certain embodiments, the invention is directed to a unit dose of a topical spray pharmaceutical formulation comprising a polymeric solution, emulsion or suspension of a hydrophilic polymer, a drug crystal precipitation inhibiting agent, an active agent, and a pharmaceutically acceptable permeation enhancer dispersed in a pharmaceutically acceptable hydroalcoholic solvent, the unit dose comprising a plurality of spray droplets, wherein 10% of the spray droplets in the unit dose have a mean diameter of about 26 μm±20 μm, about 50% of the spray droplets in the unit dose have a mean diameter of about 55 μm±20 μm, and about 90% of the spray droplets in the unit dose have a mean diameter of about 116 μm±40 μm, the unit dose topical spray provides a film surface area from about 1 cm² to about 40 cm² per spray and sets as a microporous, breathable and bioadhesive film when the hydroalcoholic solvent evaporates and provides a biphasic release of the active agent. In certain preferred embodiments, 10% of the spray droplets in the unit dose have a mean diameter of about 26 μm±1.82 μm, about 50% of the spray droplets in the unit dose have a mean diameter of about 55 μm±2.39 μm, and about 90% of the spray droplets in the unit dose have a mean diameter of about 116 μm±4.9 μm. In certain embodiments, the first peak concentration occurs at from about 0.17 to about 0.67 hours after the unit dose is sprayed onto the human subject, and the second peak concentration occurs at from about 4 to about 24 hours after the unit dose is sprayed onto the human subject. Preferably, the second peak concentration occurs at from about 9 to about 24 hours after the unit dose is sprayed onto the human subject. In certain preferred embodiments where the drug is bupivacaine hydrochloride, the first peak concentration occurs at from about 0.17 to about 0.67 hours after the unit dose is sprayed onto the human subject, and the second peak concentration occurs at from about 4 to about 24 hours after the unit dose is sprayed onto the human subject. Preferably the second peak concentration occurs at from about 9 to about 24 hours after the unit dose is sprayed onto the human subject. In certain preferred embodiments, the bupivacaine hydrochloride is present in the unit dose in an amount from about 0.5 to about 40 mg, more preferably from about 0.5 to about 20 mg. Most preferably, the bupivacaine hydrochloride is present in a supersaturated solution in the formulation. It has been found that this occurs, e.g., when the amount of bupivacaine in the unit dose is at least about 12 mg per spray.

In certain preferred embodiments, the unit dose provides a first peak concentration from about 29 pg/ml to about 380 pg/ml bupivacaine, and a second peak plasma concentration from about 864 pg/ml to about 3463 pg/ml bupivacaine.

In certain preferred embodiments, the film forming topical spray formulation of the invention comprises from about 1% to about 50% hydrophilic film forming polymer(s), from about 0.05% to about 35% active agent(s), from about 0.05% to about 50% permeation enhancer, and from about 0.01% to about 30% optional additional pharmaceutically acceptable excipients (as described herein), in a hydroalcoholic solvent. In certain preferred embodiments, the film forming topical spray formulation comprises from about 2% to about 10% by weight hydrophilic film forming polymer(s). In certain most preferred embodiments, the film forming topical spray formulation comprises from about 2.5% to about 7.5% by weight hydrophilic film forming polymer(s). In certain preferred embodiments, the film forming topical spray formulation comprises from about 1% to about 15% by weight permeation enhancer(s). In certain preferred embodiments, the film forming topical spray formulation comprises from about 2% to about 12% by weight permeation enhancer(s), and in certain embodiments most preferably from about 2.5% to about 10% permeation enhancer.

In certain embodiments, the film forming topical spray formulation further comprises from about 1% to about 35% hydrophobic polymer.

In certain preferred embodiments, the film forming topical spray formulation of the invention consists of from about 2.5% to about 20% hydrophilic film forming polymer(s), from about 0.75% to about 20% active agent(s), from about 2.5% to about 20% permeation enhancer, and from about 0.05% to about 20% optional additional pharmaceutically acceptable excipients (as described herein), in a pharmaceutically acceptable solvent.

In preferred embodiments, the solvent concentration is from about 20 to about 99%, by weight. In certain preferred embodiments, the solvent is a hydroalcoholic solvent as described herein.

The surface area to be covered by the film forming topical spray formulation will vary depending upon the area to be treated and the active agent(s), and the intended effect (e.g., topical versus systemic). In general, the film surface area is preferably from about 1 cm² to about 50 cm² per spray, and in certain embodiments more preferably from about 1 cm² to about 20 cm² per spray. In general, the number of sprays per application can vary between 1 to about 7 or between 1 to about 20 sprays. The droplet size distribution in each spray of the topical spray formulation (e.g., via a pump spray) will provide micro- and nan-droplets comprising the active agent(s), hydrophilic polymer and permeation enhancer in a (preferably) hydroalcoholic solvent. The formulation can be in solution, suspension, gel or emulsion form.

In certain preferred embodiments, the film is transparent. However, in other embodiments, the topical spray solution is colored, either due to the ingredients themselves or via the addition of a pharmaceutically acceptable colorant which may be applied to the skin and/or systemically absorbed.

In certain embodiments, the formulation may be sterilized in any pharmaceutically acceptable manner known to those skilled in the art.

### Film-Forming Polymer

The preferred film forming polymer includes an effective amount of a hydrophilic polymer to provide the desired release of active agent as described herein. The hydrophilic polymer may comprise polyvinyl pyrollidone (also known as PVP, povidone or copovidone) or a derivative thereof, polyvinyl alcohol, polyvinyl acetate, water soluble gums, water soluble celluloses (e.g., hydroxypropylmethylcellulose, hydroxypropylcellulose, hydroxyethylcellulose and the like), dextrans, hyaluronic acid, cyclodextrins, polysaccharide polymers, polyvinyl caprolactam-polyvinyl acetate-polyethylene glycol graft copolymer and combinations of any of the foregoing. In certain embodiments, the film forming polymer consists of hydrophilic polymer(s) only.

In preferred embodiments, the film forming polymer comprises from about 1% to about 50% of the topical spray formulation, and more preferably from about 2.5% to about 20% of the topical spray formulation.

In certain preferred embodiments, the film forming polymer is povidone.

In certain embodiments, part or all of the hydrophilic film forming polymer comprises or consists of hydrocolloids not limited to, synthetic gums or natural gums such as plant exudates (gum arabic, ghatti, karaya, and tragacanth); plant seed gums (guar, locust bean and acacia), seaweed extracts (agar, algin, alginate salts and carrageenin), cereal gums (starches and modified starches), fermentation or microbial gums (dextran and xanthan gum), biosynthetic gums, gelatins, pectin, casein, welan gum, gellan, rhamsan gum, synthetic gums (polyvinylpyrrolidone, low methoxyl pectin, propyleneglycol alginates, carboxymethyl locust bean gum and carboxymethyl guar gum), pullulan, other water-swellable or hydratable hydrocolloid gums known to those skilled in the art, and the like. The term hydrocolloid gum is used regardless of the state of hydration. The hydrocolloid gum can provide suitable viscosity to provide a flowable formulation that may be utilized in conjunction with the various embodiments of the invention described herein. The hydrocolloid gum utilized in the formulation of the invention may also comprise a combination of gums.

In certain preferred embodiments, the formulation further comprises from about 1% to about 50% of a drug crystal precipitation inhibiting agent which prevents or substantially prevents the drug(s) included in the formulation from precipitating. In certain preferred embodiments the formulation comprises from about 2% to about 20% of the drug crystal precipitation inhibiting agent, and in certain embodiments, more preferably from about 2.5 to about 10% of the drug crystal precipitation inhibiting agent. The amount of the drug crystal precipitation inhibiting agent may be based on visual inspection of the film for the presence of drug crystals after solvent evaporation and film uniformity. In certain embodiments and with respect to certain drugs, there appears to be a minimum threshold (e.g. 2.5%) where in spite of drug precipitation there is sufficient drug incorporated in the film that allows for permeation of a therapeutically effective dose. The choice of drug incorporated into the formulation may affect the minimum threshold of the drug crystal precipitation inhibiting agent which is preferably included in the formulation. In certain preferred embodiments, the drug crystal precipitation inhibiting agent is selected from cellulose derivatives such as hydroxypropyl methylcellulose (HPMC), hydroxypropylmethylcellulose acetate succinate (HPMCAS), methylcellulose (MC), hydroxypropyl cellulose (HPC), and vinyl polymers such as polyvinyl pyrollidone (also known as PVP, povidone or copovidone) or a derivative thereof, polyvinylalcohol (PVA), poly(acrylic acid) (PAA), polyvinylpyrrolidone vinyl acetate (PVPVA), or mixtures of any of the foregoing. In certain preferred embodiments, the hydrophilic polymer and the drug crystal precipitation inhibiting agent are comprised of the same agent, e.g., PVP.

In certain embodiments, up to about 35% of the film forming polymer may be comprised of a hydrophobic polymer. Suitable hydrophobic polymers include acrylate copolymers, methacrylic polymers and copolymers, acrylate/octylacrylamide copolymer, methyl cellulose, ethyl cellulose, methacrylic polymer, Polyurethane-14 and AMP-acrylates copolymer, Poly(butyl methacrylate, (2-dimethylaminoethyl)methacrylate, methyl methacrylate) 1:2:1, Poly(ethyl acrylate, methyl methacrylate) 2:1, Poly(ethyl acrylate, (2-trimethylaminoethyl)methacrylate, methyl methacrylate) 1:0.2:2 chloride, Poly(methacrylic acid, methyl methacrylate) 1:2, Polyisobutylene, silicon gum, silicon dioxide, microcrystalline cellulose, sodium carboxyl cellulose, and the like.

In certain preferred embodiments where the hydrophobic polymer comprises acrylic polymers or copolymers, methacrylic polymers and copolymers, including ethoxyethyl methacrylates, cynaoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers. In certain embodiments, the acrylic polymer is comprised of one or more ammonia methacrylate copolymers. Ammonia methacrylate copolymers are well known in the art, and are described in NF XVII as fully polymerized copolymers of acrylic and methacrylic acid esters with a low content of quaternary ammonium groups. Preferred film-formers include a non-ionic copolymer of methyl methacrylate and butyl methacrylate (Plastoid B®), a copolymer of dimethylamine ethyl methacrylate and a neutral methacrylic acid ester (Eudragit E100®), ammonio methacrylate copolymer type B (Eudragit RS®), ammonio methacrylate copolymer type A (Eudragit RL®), methacrylic acid copolymer type A (Eudragit L100®), methacrylic acid copolymer type B (Eudragit S100®), and the like.

The hydrophobic or hydrophobic polymers may be selected from cellulose and cellulose derivatives (such as the cellulose ethers and esters mentioned previously), starches or modified starches, as well as other pharmaceutically acceptable hydrophobic materials known to those skilled in the art. An example of a preferred hydrophobic cellulosic material is ethylcellulose. Other useful cellulose derivatives (including but not limited to hydroxymethylcellulose, carboxymethylcellulose, hydroxyethylcellulose, hydroxymethylcellulose, starch, hydroxypropylmethylcellulose, hydroxypropylcellulose, methylcellulose, hydroxypropylmethylcellulose), carboxyvinyl polymers, poly-1,4-glucans, e.g., starch glycogen, amylose, amylopectin, carboxyvinyl polymers, combinations or mixtures thereof, and the like. Useful water-soluble derivatives of poly-1,4-glucans include hydrolyzed amylopectin, hydroxyalkyl derivatives of hydrolyzed amylopectin such as hydroxyethyl starch (HES), hydroxyethyl amylose, dialdehyde starch, and the like. Suitable hydrophilic polymers include but are not limited to poly(acrylic acid), hydroxylpropyl methyl cellulose, polyvinyl pyrrolidone, polyethylene glycol, and polyethylene oxide.

In certain embodiments, the film-forming polymer can provide thixotropic behavior in use. For example, the film-forming polymer may be an amidic derivative of a carboxymethyl cellulose based thixotropic gel, and forms a homogenous three-dimensional scaffold, which maintains the thixotropic property of the (linear) polysaccharide. Amphiphilic derivatives of sodium alginate, prepared by chemical covalent binding of long alkyl chains on to the polysaccharide backbone via ester functions, form strong hydrogels in aqueous solutions with shear-thinning and thixotropic behavior. Such a hydrogel was used for the carrier or encapsulation of pharmaceutical active ingredients. In such embodiments, the thixotropic gel spray was prepared by adding the film forming polymer and thixotropic agent to the solvent and stirring the solution to ensure complete hydration and or dissolution of the polymers. The solvent used was water, hydro-alcoholic and Ethanol (95%) for preparations. Having obtained a suspended hydrated polymer gel drug, volatile solvent, permeation enhancers were added and other optional excipients. After addition of all excipients the solution was stirred to ensure complete dissolution of drug and excipients before use. The formulations were stored in glass vials sealed tightly with a cap or spray pump.

The initial permeation rate of the film forming topical spray formulation of the invention increases with decreasing polymer concentration. However, in order to obtain better film properties, a polymer concentration in certain embodiments of about 5% or greater is preferred.

### Solvents

The preferred solvent for the polymeric film forming topical spray formulation includes ethanol, isopropyl alcohol, acetone, n-butanol, methylene chloride, methylene dimethyl ether, water, hydroalcoholic system alone and or in combination of two or more solvents. The solvent concentration in the topical spray formulation can vary between about 20% to about 99% of the formulation. Preferably, the solvent is a hydroalcoholic solvent.

### Permeation Enhancers

In certain preferred embodiments, the polymeric film forming topical spray formulation further comprises an effective amount one or more permeation enhancers which allows a sufficient amount of the dose of the drug(s) to permeate through the skin. Preferred permeation enhancers include isopropyl myristate, Oleic acid, Capric acid, Lauric acid, Lauric acid, pharmaceutically acceptable glycol derivatives (e.g., propylene glycol, diethylene glycol monoethyl ether), methyloleate, lysophosphatidylcholine, phosphatidylcholine, aprotinin, azone, cyclodextrin, dextran sulfate, menthol, polysorbate 80, sulfoxides and various alkyl glycosides, urea, ethanol, caprylic alcohol, oleyl alcohol, n-methyl-2-pyrrolidone, sodium lauryl sulfate, isostearic acid, oleth-2, polyethylene glycol, polyoxyl cetostearyl ether, polyoxyl oleyl ether, polyoxyl lauryl ether, polyoxyl stearyl ether, benzyl alcohol, mixtures of any of the foregoing, and the like. In certain preferred embodiments, the permeation enhancer is selected from isopropyl myristate, oleth-2, oleic acid, 2-pyrolidone, isostearic acid, oleyly alcohol, polysorbate 80, polyethylene glycol 600, and mixtures of any of the foregoing.

In embodiments where the permeation enhancer is polyethylene glycol, all grades of polyethylene glycol are contemplated for use in preparation of the local anesthetic stock. Polyethylene glycol is available in many different grades having varying molecular weights. For example, polyethylene glycol is available as PEG 200; PEG 300; PEG 400; PEG 540 (blend); PEG 600; PEG 900; PEG 1000; PEG 1450; PEG 1540; PEG 2000; PEG 3000; PEG 3350; PEG 4000; PEG 4600 and PEG 8000. In certain embodiments the polyethylene glycol is preferably PEG 300.

In certain embodiments, the base may be a polysorbate. Polysorbates are nonionic surfactants of sorbitan esters. Polysorbates useful in the present invention include, but are not limited to polysorbate 20, polysorbate 40, polysorbate 60, polysorbate 80 (Tween 80) and any combinations or mixtures thereof. In certain preferred embodiments, polysorbate 80 may be utilized as the pharmaceutically acceptable permeation enhancer.

It is recognized that the permeation enhancer may also act as a plasticizer. In this regard, the polymeric film forming topical spray formulation may further comprise one or more plasticizers. Suitable plasticizers include but are not limited to propylene glycol, polyethylene glycol, citrate esters, dimethyl isosorbide, castor oil, and combinations of any of the foregoing.

In the case of thinner film thickness, permeation enhancer concentration becomes more significant for the initial permeation rate of active through membrane or skin. The thinner film thickness alternatively represents film spread over a larger surface area on human skin after spray administration.

### Optional Excipients

In addition to the active agent(s) (e.g., local anesthetic), the polymeric film forming topical spray formulation may additionally include physiologically acceptable components such as sodium chloride and like materials conventionally used to achieve isotonicity with typical body fluids, pH buffers to establish a physiologically compatible pH range and to enhance the solubility of the anesthetic present, vasoconstrictors such as epinephrine, preservatives, stabilizers and antioxidants and the like.

In certain other embodiments, an additional surfactant (co-surfactant) and/or buffering agent can preferably be combined with one or more of the pharmaceutically acceptable vehicles previously described herein so that the surfactant and/or buffering agent maintains the product at an optimal pH for stability. The surfactant and/or buffering agent may also prevent the initial stinging or burning discomfort associated with administration of the active agent on the skin (e.g, local anesthetic).

In certain other embodiments, an additional anti-oxidant and/or stabilizing agent can preferably be combined with one or more of the pharmaceutically acceptable vehicles previously described herein so that the anti-oxidant and/or stabilizing agent maintains the drug product at an optimal impurity level for stability. The anti-oxidant and/or stabilizing agent also prevents the initial degradation of active agent during the manufacturing process. The anti-oxidant may be selected, e.g., from ascorbic acid, EDTA, trolamine, tocopherol, propyl galate, sodium sulfite and mixtures of any of the foregoing.

The formulation of the invention may contain preservatives to prevent microbial growth. Suitable preservatives for use in the present invention include, but are not limited to benzoic acid, boric acid, p-hydroxybenzoates, phenols, chlorinated phenolic compounds, alcohols, quarternary compounds, mercurials, mixtures of the foregoing and the like.

The composition may further comprise one or more additional components selected from the group consisting of solubilizers, plasticizers, and water soluble additives.

Preferred plasticizers include triethyl citrate, dimethyl isosorbide, acetyltributyl citrate, castor oil, propylene glycol, and polyethylene glycol, or any two or more of the above in combination.

### Viscosity of the Formulation

In most applications, optimal viscosities of the system of the invention will range from about 10 to about 2,000,000 centipoise, preferably from about 0.3 to 1000 centipoise, and more preferably from about 0.5 to about 100 centipoise, at 37° C. While the benefit of the invention is realized over a broad range of elevated viscosities, the optimal viscosities will be different for different applications. The desired viscosity for any given formulation or use may vary, for example, according to the preference of the physician, the manner of application and type of applicator used, the amount of formulation needed, the area to which the formulation is to be applied, and similar considerations.

### Active Agents

The amount of drug permeating through the membrane is directly proportional to concentration of active (drug) in the formulation, with the greater amount of drug concentration in the formulation leading to greater cumulative drug permeation.

In certain preferred embodiments of the invention, the active agent comprises a local anesthetic. Examples of local anesthetic agents useful in the gel formulations of the invention include amide type local anesthetics, such as mepivacaine, lidocaine, mepivacaine, etidocaine and prilocaine; ester type local anesthetics, such as procaine, chloroprocaine, and tetracaine; and antihistamine-like anesthetics, such as benadryl. These anesthetics can be present in the anesthetic pharmaceutical combination alone or as a mixture of two or more thereof. Thus, examples of useful local anesthetics are lidocaine, bupivacaine, dibucaine, tetracaine, etidocaine, mepivacaine, ropivacaine, benzocaine, ambucaine, amylocaine, butamben, 2-chloroprocaine, cyclomethycaine, ethyl aminobenzoate, euprocin, levoxadrol, orthocaine, piperocaine and parethoxycaine. In certain preferred embodiments, the local anesthetic is bupivacaine, ropivacaine, dibucaine, procaine, chloroprocaine, prilocaine, mepivacaine, etidocaine, tetracaine, lidocaine, and xylocaine, or mixtures thereof. In certain preferred embodiments, the local anesthetic is lidocaine, bupivacaine, levo-bupivacaine, ropivacaine, tetracaine, mepivacaine, prilocaine, benzocaine, editocaine, or combinations of any of the foregoing. The phrase “local anesthetic” also can include drugs of a different class than those traditionally associated with local anesthetic properties, such as morphine, fentanyl, and agents which, for example, can provide regional blockade of nociceptive pathways (afferent and/or efferent). Other compounds which may be used as a local anesthetic in the gel formulations of the invention include antihistamine-like anesthetics, such as benadryl. Phenol may also be used as the local anesthetic. Those skilled in the art will recognize other agents which have been recognized to possess local anesthetic properties, such as the substituted piperidines and pyrollidines described in U.S. Pat. No. 4,302,465 (Aberg, et al.) and the aminoindane piperidine compounds described in U.S. Pat. No. 6,413,987 (Aberg, et al.), both of which patents are hereby incorporated by reference. The term local anesthetic is also deemed for purposes of the present invention to encompass the local anesthetic base or a pharmaceutically acceptable salt, polymorph, complex or pro-drug thereof. Many other examples of both drugs and local anesthetics will be readily apparent to those skilled in the art, and are considered to be encompassed by this disclosure and appended claims.

The local anesthetic can be in the form of a salt, for example, the hydrochloride, bromide, acetate, citrate, carbonate, sulfate or phosphate. In certain embodiments, the local anesthetic agent is in the form of a free base. Local anesthetics can be in the form of a salt, for example, the hydrochloride, bromide, acetate, citrate, carbonate or sulfate, or in the form of a free base. Many of the local anesthetics are conventionally used in the form of their acid addition salts, as this provides solubility in aqueous injection media. In certain embodiments of the invention, it is desirable to use the local anesthetics in free base form, or with only a small proportion of the acid addition salt of the local anesthetic present (addition of small quantities of the acid addition salt may provide enhanced release if desired). The free base generally provides a slower initial release and avoids an early “dumping” of the local anesthetic at the injection site. Preferred local anesthetic agents include, e.g., bupivacaine, ropivacaine or lidocaine. For bupivacaine, the free base provides a slower initial release and avoids an early “dumping” of the local anesthetic at the site of administration. Also, it has been found that the formulations provide dose proportionality with the bupivacaine salt (i.e., bupivacaine hydrochloride), but not with the base form. Other local anesthetics may act differently.

In certain preferred embodiments, the dose of local anesthetic contained in a unit dose is from about from about 0.01 mg to about 50 mg bupivacaine. In other preferred embodiments, the dose is from about 0.1 mg to about 20 mg bupivacaine, and preferably from about 1 mg to about 10 mg or to about 20mg bupivacaine.

In other embodiments, the active agent is an anesthetic such as a barbiturate (e.g., amobarbital, methohexital, thiamylal, thiopental), a benzodiazepine (e.g., diazepam, lorazepam, midazolam), or etomidate, ketamine, or propofol.

In certain embodiments of the invention, the film forming topical spray formulation provides a systemic effect when applied or sprayed onto an environment of use (e.g., human skin). Any active ingredient (drug) for which it is useful to combine the advantages of a pulsatile plasma profile may be used in practice of the present invention, including but not limited to active agents whose pharmacological and/or therapeutic effects benefit from having a wash-out period between plasma concentration peaks, such as those active ingredients susceptible to the development of patient tolerance. Example active ingredients (drugs) include but are not limited to peptides or proteins, hormones, analgesics, anti-migraine agents, anti-coagulant agents, narcotic antagonists, chelating agents, anti-anginal agents, chemotherapy agents, sedatives, anti-neoplastics, prostaglandins and antidiuretic agents, drug compounds acting on the central nervous system such as cerebral (central nervous system) stimulants, for example methylphenidate; pain management active ingredients; alkaloids such as opiates, for example morphine; cardiovascular drugs, such as nitrates; and agents for treating rheumatic conditions. It should be further appreciated that the present invention may be used to deliver a number of drugs including, but not limited to, peptides, proteins or hormones such as insulin, calcitonin, calcitonin gene regulating protein, growth factor (somatomedins), luteinizing hormone releasing hormone (LHRH), tissue plasminogen activator (TPA), growth hormone releasing hormone (GHRH), oxytocin, estradiol, growth hormones, leuprolide acetate, factor VIII, interleukins such as interleukin-2, and analogues thereof; analgesics such as fentanyl, sufentanil, butorphanol, buprenorphine, levorphanol, morphine, hydromorphone, hydrocodone, oxymorphone, methadone, lidocaine, bupivacaine, diclofenac, naproxen, paverin, and analogues thereof; anti-migraine agents such as sumatriptan, ergot alkaloids, and analogues thereof; anti-coagulant agents such as heparin, hirudin, and analogues thereof; anti-emetic agents such as scopolamine, ondansetron, domperidone, metoclopramide, and analogues thereof; cardiovascular agents, anti-hypertensive agents and vasodilators such as diltiazem, clonidine, nifedipine, verapamil, isosorbide-5-mononitrate, organic nitrates, agents used in treatment of heart disorders, and analogues thereof; sedatives such as benzodiazepines, phenothiozines, and analogues thereof; chelating agents such as deferoxamine, and analogues thereof; anti-diuretic agents such as desmopressin, vasopressin, and analogues thereof; anti-anginal agents such as nitroglycerine, and analogues thereof; anti-neoplastics such as fluorouracil, bleomycin, and analogues thereof; prostaglandins and analogues thereof; and chemotherapy agents such as vincristine, and analogues thereof.

In certain preferred embodiments, the active agent may be an opioid analgesic(s), including but not limited to buprenorphine, morphine, oxycodone, hydromorphone, codeine, oxymorphone, fentanyl, sufentanyl, tramadol, and hydrocodone.

In other preferred embodiments, the active agent may be a non-opioid analgesic including but limited to non-steroidal anti-inflammatory agents such as ibuprofen, naproxen, diclofenac, naproxen, ketoprofen, ketorolac, and the like.

The active agent may also be an anti-bacterial agent including, but not limited to mupirocin, bacitracin, neomycin, penicillins, cephalosporins, vancomycin, bacitracin, cephalosporins, polymxyins, amikacin, doxycycline, nystatin, amphotericin-B, tetracyclines, chloramphenicol, erythromycin, minocycline, gentamicin, neomycin, streptomycin, kanamycin, gentamicin, tobramycin, clindamycin, rifampin, nalidixic acid, flucytosine, griseofulvin, clindamycin, doxycycline, mixtures of any of the foregoing, and the like.

The active agent may also be an antiviral agent including but not limited to vidarabine, acyclovir, ribavirin, amantadine hydrochloride, interferons, dideoxyuridine, mixtures of any of the foregoing and the like.

The active agent may also be an antifungal agent including but not limited to butoconazole, tetraconazole, nystatin, miconazole, tolnaftate, undecyclic acid and its salts, mixtures of the foregoing and the like.

The active agent may alternatively be an antiparasitic agent including but not limited to quinacrine, chloroquine, quinine, mixtures of the foregoing and the like.

The active agent may also be a steroidal anti-inflammatory agents including but not limited to hydrocortisone, prednisone, fludrocortisone, triamcinolone, dexamethasone, betamethasone, desoximetasone, fluticasone mixtures of the foregoing and the like.

The active agent may also be an antihistamine (H2 antagonist) including, but not limited to diphenhydramine, chlorpheneramine, chlorcyclizine, promethazine, cimetidine, terfenadine, mixtures of the foregoing and the like.

Other preferred pharmaceutical active agents (drugs) which can be incorporated into the formulations of the invention include epinephrine, clonidine, methylphenidate, nicotine, nitroglycerin, oxybutynin, rotigotine, selegiline, scopolamine, ciclopirox, misoprostol, hyoscyamine, atropine, silver sulfadiazine, sulfanilamide, clotrimazole, terbinafine, ketoconazole, acyclovir, minoxidil, tretinoin, azelaic acid, benzoyl peroxide, capsaicin, clobetasol, desonide, miconazole, tacrolimus, salicylic acid, terbenafine, clobetasol, sumatriptan, zolmitriptan, triamcinolone, zolpidem, rivastigmine, piroxicam, amytriptylene, meloxicam, dalfampridine, spiro-oxindole compounds, gabapentin, ketamine, docosanol, pirfenidone, isotretinoin, and papaya enzymes. The active agents (drugs) can be in the base form, or can be pharmaceutically acceptable salts, complexes or derivatives of the active agent.

The formulations of the invention can include two or more of the above-mentioned ingredients (drugs) or pharmaceutically acceptable salts, complexes or derivatives thereof, as well.

In certain preferred embodiments, the active agent(s) (drug(s)) is a steroid, such as estrogen, estradiol, norethindrone, levonorgestrel, ethinylestradiol, norelgestromin, testosterone, and mixtures thereof.

In certain preferred embodiments, the active agent(s) (drug(s)) is a combination of therapeutically effective amounts of a local anesthetic (e.g., bupivacaine) and ketamine and or amitriptyline for use in treating neuropathic pain. In certain embodiments, the active agent comprises a combination of bupivacaine hydrochloride and a second active agent selected from the group consisting of ketamine, amitriptyline, and combinations thereof.

The composition contains preferably up to about 30% of the at least one medicament (drug), more preferably up to about 10% of the at least one drug and most preferably up to about 5% of the at least one drug.

The compositions are preferably in a form suitable for application by spraying from an aerosol or pump spray container.

### Treatment of Pain

Neuropathic pain is a persistent pain condition that develops secondary to nerve injury. The two most common types of peripheral neuropathic pain are post-herpetic neuralgia (PHN) and painful peripheral diabetic neuropathy (PDN). PHN is a serious complication of herpes zoster or shingles, which occurs in some patients secondary to reactivation of varicella-zoster virus residing in the dorsal root ganglia of individuals following primary infection (chicken pox) often in childhood. The main symptoms experienced during the acute period of herpes zoster infection are pain and skin rash. The pain that emerges subsequent to the healing of rash and which persists for more than three months is known as PHN. The incidence of PHN among herpes zoster patients is approximately 10-34% with a higher incidence in elderly patients (>60 years), 50% of whom are likely to develop PHN.

At present, there are no treatments that can reverse ‘neuropathic nerve injury’. Therefore treatments are strictly palliative, targeted to the provision of symptomatic pain relief with the treatment goal to reduce pain to tolerable levels. The current available pharmacological treatments for the treatment of symptomatic relief of PHN include antidepressants, serotonin noradrenaline reuptake inhibitors (SNRIs), selective serotonin reuptake inhibitors (SSRIs), anti-epileptics, opioids, N-Methyl D-Aspartate (NMDA) receptor antagonists and topical anesthetics like lidocaine, bupivacaine, ropivacaine. Lidocaine 5% patch is a topical agent that has been approved by the FDA for symptomatic relief of PHN. The primary mechanism of action is through lidocaine inhibiting voltage-gated Na+ channels in damaged peripheral nerves that have developed pathological spontaneous activity, which is thought to be associated with injury-induced increased expression of Na+ channels.

Lidocaine (xylocaine) was introduced as a local anesthetic in 1948. Local anesthetics act by preventing the generation and conduction of nerve impulse. Their primary site of action is the cell membrane. They block conduction by decreasing or preventing the large transient increase in the permeability of excitable membranes to Na+ that normally is produced by a slight depolarization of the membrane. This action of local anesthetics is due to their direct interaction with voltage gated Na+ channels. As the anesthetic action progressively develops in a nerve, the threshold for electrical excitability gradually increases, the rate of rise of the action potential declines, impulse conduction slows, and the safety factor for conduction decreases; these factors decrease the probability of propagation of the action potential, and nerve conduction fails. Bupivacaine, a longer acting variant of lidocaine is a preferred local analgesic.

The degree of block produced by a given concentration of local anesthetic depends on how the nerve has been stimulated and on its resting membrane potential. Thus, a resting nerve is much less sensitive to a local anesthetic than is one that is repetitively stimulated; higher frequency of stimulation and more positive membrane potential cause a greater degree of anesthetic block. These frequency and voltage dependent effects of local anesthetics occur because the local anesthetic molecule in its charged form gains access to its binding site within the pore only when the Na+ channel is in an open state and because the local anesthetic molecule binds more tightly to and stabilizes the inactivated state of the Na+ channel. Local anesthetics exhibit these properties to different extents depending on their pKa, lipid solubility and molecular size.

Lidoderm patch is cumbersome to use. It is supplied as a 10 cm×14 cm patch. The patient is instructed to apply three patches to the most painful area once for up to 12 hours. Per the package insert, the site of patch application may develop erythema, edema, bruising, papules, vesicles, discoloration, depigmentation, burning sensation, pruritus and abnormal sensation which is reversed upon patch removal. The patch is not patient friendly more so when the pain presents itself outside the upper body and trunk area specifically in the facial area (myofascial pain). It is anticipated that the present invention, which is an easy to use proprietary dermal spray formulation to overcome many of the disadvantages of patch application while still providing pain relief in patients with PHN. A topical spray formulation would provide application convenience.

Accordingly in certain preferred embodiments, the topical spray formulation of the invention is used for treating general pain, neuropathic pain neuropathic pain (e.g., erythromelalgia, post-herpetic neuralgia (PHN), fibromyalgia and/or complex regional pain syndrome (CRPS), among others), postoperative pain, sports pain, osteoporosis pain, pain resulting from cosmetic procedures, dental pain, wound pain and burn pain.

In certain preferred embodiments, the topical spray formulation of the invention is used to reduce pain during the treatment of or amelioration of symptoms of any one or more of the following diseases which cause neuropathic pain or which have a neuropathic pain component: Abdominal Wall Defect, Abdominal Migraine, Achondrogenesis, Achondrogenesis Type IV, Achondrogenesis Type III, Achondroplasia, Achondroplasia Tarda, Achondroplastic Dwarfism, Acquired hnmunodeficiency Syndrome (AIDS), Acute Intermittant Porphyria, Acute Porphyrias, Acute Shoulder Neuritis, Acute Toxic Epidermolysis, Adiposa Dolorosa, Adrenal Neoplasm, Adrenomyeloneuropathy, Adult Dermatomyositis, Amyotrophic Lateral Sclerosis, Amyotrophic Lateral Sclerosis-Polyglucosan Bodies, AN, AN 1, AN 2, Anal Rectal Malformations, Anal Stenosis, Arachnitis, Arachnoiditis Ossificans, Arachnoiditis, Arteritis Giant Cell, Arthritis, Arthritis Urethritica, Ascending Paralysis, Astrocytoma Grade I (Benign), Astrocytoma Grade II (Benign), Athetoid Cerebral Palsy, Barrett Esophagus, Barrett Ulcer, Benign Tumors of the Central Nervous System, Bone Tumor-Epidermoid Cyst-Polyposis, Brachial Neuritis, Brachial Neuritis Syndrome, Brachial Plexus Neuritis, Brachial-Plexus-Neuropathy, Brachiocephalic Ischemia, Brain Tumors, Brain Tumors Benign, Brain Tumors Malignant, Brittle Bone Disease, Bullosa Hereditaria, Bullous CIE, Bullous Congenital Ichthyosiform Erythroderma, Bullous Ichthyosis, Bullous Pemphigoid, Burkitt's Lymphoma, Burkitt's Lymphoma African type, Burkitt's Lymphoma Non-african type, Calcaneal Valgus, Calcaneovalgus, Cavernous Lymphangioma, Cavernous Malformations, Central Form Neurofibromatosis, Cervical Spinal Stenosis, Cervical Vertebral Fusion, Charcot's Disease, Charcot-Marie-Tooth, Charcot-Marie-Tooth Disease, Charcot-Marie-Tooth Disease Variant, Charcot-Marie-Tooth-Roussy-Levy Disease, Childhood Dermatomyositis, Chondrodysplasia Punctata, Chondrodystrophia Calcificans Congenita, Chondrodystrophia Fetalis, Chondrodystrophic Myotonia, Chondrodystrophy, Chondrodystrophy with Clubfeet, Chondrodystrophy Epiphyseal, Chondrodystrophy Hyperplastic Form, Chondroectodermal Dysplasias, Chondrogenesis Imperfecta, Chondrohystrophia, Chondroosteodystrophy, Chronic Adhesive Arachnoiditis, Chronic Idiopathic Polyneuritis (CIP), Chronic Inflammatory Demyelinating Polyneuropathy, Chronic Inflammatory Demyelinating Polyradiculoneuropathy, Cicatricial Pemphigoid, Complex Regional Pain Syndrome, Congenital Cervical Synostosis, Congenital Dysmyelinating Neuropathy, Congenital Hypomyelinating Polyneuropathy, Congenital Hypomyelination Neuropathy, Congenital Hypomyelination, Congenital Hypomyelination (Onion Bulb) Polyneuropathy, Congenital Ichthyosiform Erythroderma, Congenital Tethered Cervical Spinal Cord Syndrome, Cranial Arteritis, Crohn's Disease, Cutaneous Porphyrias, Degenerative Lumbar Spinal Stenosis, Demyelinating Disease, Diabetes Mellitus Diabetes Insulin Dependent, Diabetes Mellitus, Diabetes Mellitus Addison's Disease Myxedema, Discoid Lupus, Discoid Lupus Erythematosus, Disseminated Lupus Erythematosus, Disseminated Neurodermatitis, Disseminated Sclerosis, EDS Kyphoscoliotic, EDS Kyphoscoliosis, EDS Mitis Type, EDS Ocular-Scoliotic, Elastosis Dystrophica Syndrome, Encephalofacial Angiomatosis, Encephalotrigeminal Angiomatosis, Enchondromatosis with Multiple Cavernous Hemangiomas, Endemic Polyneuritis, Endometriosis, Eosinophilic Fasciitis, Epidermolysis Bullosa, Epidermolysis Bullosa Acquisita, Epidermolysis Bullosa Hereditaria, Epidermolysis Bullosa Letalias, Epidermolysis Hereditaria Tarda, Epidermolytic Hyperkeratosis, Epidermolytic Hyperkeratosis (Bullous CIE), Familial Lumbar Stenosis, Familial Lymphedema Praecox, Fibromyalgia, Fibromyalgia-Fibromyositis, Fibromyositis, Fibrositis, Fibrous Ankylosis of Multiple Joints, Fibrous Dysplasia, Fragile X syndrome, Generalized Fibromatosis, Guillain-Barre Syndrome, Hemangiomatosis Chondrodystrophica, Hereditary Sensory and Autonomic Neuropathy Type I, Hereditary Sensory and Autonomic Neuropathy Type II, Hereditary Sensory and Autonomic Neuropathy Type III, Hereditary Sensory Motor Neuropathy, Hereditary Sensory Neuropathy type I, Hereditary Sensory Neuropathy Type I, Hereditary Sensory Neuropathy Type II, Hereditary Sensory Neuropathy Type III, Hereditary Sensory Radicular Neuropathy Type I, Hereditary Sensory Radicular Neuropathy Type I, Hereditary Sensory Radicular Neuropathy Type II, Herpes Zoster, Hodgkin Disease, Hodgkin's Disease, Hodgkin's Lymphoma, Hyperplastic Epidermolysis Bullosa, Hypertrophic Interstitial Neuropathy, Hypertrophic Interstitial Neuritis, Hypertrophic Interstitial Radiculoneuropathy, Hypertrophic Neuropathy of Refsum, Idiopathic Brachial Plexus Neuropathy, Idiopathic Cervical Dystonia, Juvenile (Childhood) Dermatomyositis (JDMS), Juvenile Diabetes, Juvenile Rheumatoid Arthritis, Pes Planus, Leg Ulcer, Lumbar Canal Stenosis, Lumbar Spinal Stenosis, Lumbosacral Spinal Stenosis, Lupus, Lupus, Lupus Erythematosus, Lymphangiomas, Mononeuritis Multiplex, Mononeuritis Peripheral, Mononeuropathy Peripheral, Monostotic Fibrous Dysplasia, Multiple Cartilaginous Enchondroses, Multiple Cartilaginous Exostoses, Multiple Enchondromatosis, Multiple Myeloma, Multiple Neuritis of the Shoulder Girdle, Multiple Osteochondromatosis, Multiple Peripheral Neuritis, Multiple Sclerosis, Musculoskeletal Pain Syndrome, Neuropathic Amyloidosis, Neuropathic Beriberi, Neuropathy of Brachialpelxus Syndrome, Neuropathy Hereditary Sensory Type I, Neuropathy Hereditary Sensory Type II, Nieman Pick disease Type A (acute neuronopathic form), Nieman Pick disease Type B, Nieman Pick Disease Type C (chronic neuronopathic form), Non-Scarring Epidermolysis Bullosa, Ochronotic Arthritis, Ocular Herpes, Onion-Bulb Neuropathy, Osteogenesis Imperfect, Osteogenesis Imperfecta, Osteogenesis Imperfecta Congenita, Osteogenesis Imperfecta Tarda, Peripheral Neuritis, Peripheral Neuropathy, Perthes Disease, Polyarteritis Nodosa, Polymyalgia Rheumatica, Polymyositis and Dermatomyositis, Polyneuritis Peripheral, Polyneuropathy Peripheral, Polyneuropathy and Polyradiculoneuropathy, Polyostotic Fibrous Dysplasia, Polyostotic Sclerosing Histiocytosis, Postmyelographic Arachnoiditis, Primary Progressive Multiple Sclerosis, Psoriasis, Radial Nerve Palsy, Radicular Neuropathy Sensory, Radicular Neuropathy Sensory Recessive, Reflex Sympathetic Dystrophy Syndrome, Relapsing-Remitting Multiple Sclerosis, Sensory Neuropathy Hereditary Type I, Sensory Neuropathy Hereditary Type II, Sensory Neuropathy Hereditary Type I, Sensory Radicular Neuropathy, Sensory Radicular Neuropathy Recessive, Sickle Cell Anemia, Sickle Cell Disease, Sickle Cell-Hemoglobin C Disease, Sickle Cell-Hemoglobin D Disease, Sickle Cell-Thalassemia Disease, Sickle Cell Trait, Spina Bifida, Spina Bifida Aperta, Spinal Arachnoiditis, Spinal Arteriovenous Malformation, Spinal Ossifying Arachnoiditis, Spinal Stenosis, Stenosis of the Lumbar Vertebral Canal, Still's Disease, Syringomyelia, Systemic Sclerosis, Talipes Calcaneus, Talipes Equinovarus, Talipes Equinus, Talipes Varus, Talipes Valgus, Tandem Spinal Stenosis, Temporal Arteritis/Giant Cell Arteritis, Temporal Arteritis, Tethered Spinal Cord Syndrome, Tethered Cord Malformation Sequence, Tethered Cord Syndrome, Tethered Cervical Spinal Cord Syndrome, Thalamic Pain Syndrome, Thalamic Hyperesthetic Anesthesia, Trigeminal Neuralgia, Variegate Porphyria, Vertebral Ankylosing Hyperostosis amongst others. These conditions may be treated with one or more local anesthetics as described herein.

For purposes of the present invention, in certain embodiments the method of treatment induces an analgesic response to neuropathic and/or inflammatory pain being suffered by a mammalian, preferably human, patient. A patient, in this context, is also referred to as a “subject”, “target” or “recipient”. In this context the terms “analgesia” and “analgesic response” are intended to describe a state of reduced sensibility to pain at the site of application in certain embodiments, and systemically in other embodiments. To assess the level of reduction of sensibility to pain associated with the analgesia induced by the methods according to the present invention it is possible to conduct tests such as a VAS (visual analogue scales for pain intensity) questionnaire or the short form McGill pain questionnaire and/or verbal rating scales for pain intensity and/or measurement of tactile allodynia using von Frey hairs or similar device. These tests are standard tests within the art and would be well known to the skilled person.

### Local Anesthetic Embodiment

In certain preferred embodiments, the topical spray formulation of the invention includes a local anesthetic as an active agent. It is especially preferred in certain embodiments that the local anesthetic is bupivacaine hydrochloride. It has been unexpectedly found that the topical spray formulation is dose proportional only when the bupivacaine salt is used; the bupivacaine base incorporated into the topical spray compositions of the invention are not dose proportional.

In certain preferred embodiments, the bupivacaine hydrochloride topical spray formulation comprises a sufficient concentration of bupivacaine such that a dose from about 0.1 mg to about 50 mg is applied to the skin of a patient in a desired location. In certain preferred embodiments, the dose of bupivacaine is about 10 mg, based on the bupivacaine hydrochloride salt. The dosage regimen can be, for example, 5 sprays (each spray containing 10 mg bupivacaine hydrochloride), in 5 distinct areas. In other preferred embodiments, from about 1 to about 30, or from about 1 to about 10, and preferably from about 3 to about 20 sprays of the topical spray formulation can be sprayed onto the skin about every 6 hours, twice a day, three times a day, once a day, or as needed.

Provides an in-vitro release of the bupivacaine of at least 1.0% after 2 hours, when tested via an in-vitro permeation study performed on a Strat-M synthetic membrane using In-line PermeGear ILC07 automatic diffusion cell system.

### DETAILED DESCRIPTION OF PREFERRED EMBODIMENTS

The following examples in accordance with the present invention are not to be construed as limiting the present invention in any manner and are only samples of the various formulations described herein.

### Example 1

In Examples 1, a bupivacaine HCl topical spray formulation was prepared using the ingredients set forth in Table 1.

Film forming sprays were prepared by adding target drug, non-volatile solvent and permeation enhancer to the solvent while stirring the solution to ensure complete dissolution of the drug and other excipients. The solvent used was ethanol (95%). Having obtained a clear solution, polymer were added and other optional excipients. After addition of all excipients the solution was stirred to ensure complete dissolution and or hydration of polymer prior to use. The formulation was stored in glass vials sealed tightly with a cap or spray pump.

TABLE 1

| | Example 1 |
|---|---|
| | mg/spray |
| Bupivacaine HCl | 2.00 |
| Plastoid B | 6.00 |
| Eudragit EPO | 0.50 |
| Propylene Glycol | 5.00 |
| Transcutol P | 10.00 |
| Ethanol 95% | qs |
| Isopropyl Alcohol | qs |
| Menthol | 0.05 |
| | 100.00 |

The process for preparing the formulations is as follows:

Add drug, propylene glycol, Transcutol® (highly purified diethylene glycol monoethyl ether commercially available from Gattefosse; it is a highly purified hydrophilic solvent for drugs), Tween® 80 (polysorbate 80, a nonionic surfactant and emulsifier; Example 1), and menthol in ethanol with stirring and mix till clear solution formed. Add polymers (Plastoid B® and Eudragit® EPO) in isopropyl alcohol or water with stilling until uniform dispersion formed. Plastoid B® is a non-ionic copolymer of methyl methacrylate and butyl methacrylate, commercially available from Evonik. Eudragit® EPO is a cationic copolymer based on dimethylaminoethyl methacrylate, butyl methacrylate and methyl methacrylate, commercially available from Evonik. Add the drug clear solution in the polymeric dispersion under stirring till polymer dissolve or hydrate completely and form solution and or emulsion. Store the polymeric solution in glass bottle with cap or mechanical pump. The Eudragit EPO and Plastoid B were included as the polymers and ethanol and isopropyl alcohol were included as the solvent. 2 mg aqueous solution of active substance was used as a positive control. The films were tested in in-vitro Franz Cell Strat-M synthetic membrane experiments. The results are set forth in Table 2 below:

TABLE 2
In-vitro cumulative drug permeation (ug/cm²)

| | | Control (2 mg in |
|---|---|---|
| Time (Hr) | Example 1 | water/spray) |
| 0.5 | 0.98 | 1.33 |
| 1 | 6.17 | 34.35 |
| 2 | 10.73 | 101.92 |
| 4 | 15.72 | 167.49 |
| 6 | 20.83 | 197.59 |
| 24 | 32.77 | 219.40 |

It was observed that using the hydrophobic polymer such as Eudragit, about 1.2% of active permeates through the synthetic membrane over 24 hours. This is much lower than the amount of active that permeates from the aqueous control. The film that forms after spraying on the skin was peelable in nature and did not fully adhere to the skin or application site. Based on the film properties and rate of permeation it was decided not to use (solely) hydrophobic polymer in future studies.

### Example 2-5

In Examples 2-5, bupivacaine HCl topical spray formulations were prepared using the ingredients set forth in Table 3, and further studies were performed using the hydrophilic polymers Avicel RC-591 (sodium CMC and MCC) and povidone K-30 (PVP) with water and or ethanol as solvent.

The process for preparing the formulations is as follows:

Add Avicel® RC591 (microcrystalline cellulose and carboxymethylcellulose sodium NF, Ph. Eur., commercially available from FMC Biopolymer) (except Example 5) and povidone K30 (polyvinylpyrollidone) in purified water with stirring and mix till polymer hydrates completely. Add drug, propylene glycol, transcutol (except Example 4), permeation enhancer and menthol in ethanol or purified water with stirring and mix till clear solution formed. Add the clear solution with drug in the polymeric dispersion while stirring till drug solution disperses uniformly with thixotropic polymeric gel. Store the polymeric thixotropic gel in glass bottle with cap or mechanical pump.

The films were tested in In-vitro Franz Cell Strat-M synthetic membrane experiments. The results are set forth in Table 4 below:

TABLE 4
In-vitro cumulative drug permeation (ug/cm²)

| | | | | | Control | Control |
|---|---|---|---|---|---|---|
| | | | | | (2 mg | (2 mg in |
| Time | | | Example | Example | in water/ | ethanol/ |
| (Hr) | Lot 10 | Lot 11 | 3 | 2 | spray) | spray) |
| 0.5 | 0.33 | 0.30 | 3.22 | 2.68 | 1.33 | 13.94 |
| 1 | 0.50 | 0.31 | 5.15 | 10.97 | 34.35 | 97.09 |
| 2 | 0.75 | 0.49 | 9.18 | 76.94 | 101.92 | 483.12 |
| 4 | 0.89 | 0.64 | 18.37 | 371.43 | 167.49 | 1536.99 |
| 6 | 1.03 | 0.75 | 24.65 | 745.26 | 197.59 | 1828.75 |
| 24 | 1.65 | 1.04 | 100.65 | 1891.80 | 219.40 | 2008.52 |

From data obtained, it was concluded that if povidone is used as the polymer in the formulation containing a hydro-alcoholic solvent, active permeates at a much faster rate and extent than Avicel RC591. Avicel RC591 is composed of carboxymethylcellulose sodium which forms a gel like consistency and produces a larger droplet size upon spraying with a mechanical pump. It was concluded that using ethanol as solvent provides better permeation characteristics than water and a hydrophilic polymer is better than hydrophobic polymer with respect to permeation and adhesion to the skin.

### Example 6-14

Based on the results set forth for Examples 1-5, it was decided that ethanol will be used as solvent and different permeation enhancers and hydrophilic polymers will be evaluated in the next series of studies. Eight different types of permeation enhancers (isopropyl myristate, oleth-2, oleic acid, 2-pyrolidone, isostearic acid, oleyly alcohol, polysorbate 80 and polyethylene glycol 600) were evaluated to understand the rate and extent of drug permeation. Bupivacaine topical spray formulations were prepared using the ingredients set forth in Table 5.

The process for preparing the formulations is as follows:

Add drug, propylene glycol, oleyl alcohol and menthol in ethanol under stirring and mix till clear solution formed. Add the film-forming polymer in the clear drug solution while stirring till polymer dissolves or hydrates completely. Store the polymeric film forming solution in glass bottle with cap or mechanical pump. Each of Examples 6-14 contain 5% w/w PVP in the formulation.

The films were tested in In-vitro Franz Cell Strat-M synthetic membrane experiments. The results are set forth in Table 6 below:

TABLE 6
In-vitro cumulative drug permeation (ug/cm²)

| Time (Hr) | Ex 6 | Ex 7 | Ex 8 | Ex 9 | Ex 10 |
|---|---|---|---|---|---|
| 0.5 | 2.00 | 0.67 | 4.02 | 1.09 | 2.32 |
| 1 | 8.98 | 16.25 | 28.40 | 7.93 | 19.24 |
| 2 | 41.97 | 144.56 | 203.16 | 31.28 | 163.27 |
| 4 | 184.46 | 373.76 | 627.74 | 129.54 | 489.27 |
| 6 | 411.02 | 522.22 | 761.61 | 300.87 | 533.55 |
| 24 | 1004.05 | 1055.67 | 1059.61 | 997.45 | 638.24 |
| Time (Hr) | Ex 11 | Ex 12 | Ex 13 | Ex 14 | Control |
| 0.5 | 1.70 | 3.50 | 1.86 | 2.00 | 6.19 |
| 1 | 13.58 | 34.59 | 9.60 | 6.79 | 65.39 |
| 2 | 97.27 | 246.67 | 45.61 | 51.55 | 324.06 |
| 4 | 309.40 | 603.63 | 219.35 | 263.30 | 606.76 |
| 6 | 417.21 | 733.71 | 506.66 | 451.59 | 785.01 |
| 24 | 817.85 | 1010.53 | 1327.51 | 981.21 | 1184.22 |

Based on data obtained, it can be concluded that permeation enhancer is very critical for initial permeation of active in the first two hours. Example 6 without any permeation enhancer permeates only 50 ug/cm² of active in the first two hours and Example 12 permeates about 250 ug/cm² active in the first 2 hours. Also the type of permeation enhancer used is critical in achieving higher initial permeation of active.

### Examples 15-20

Based on the data obtained for Examples 6-14, oleyl alcohol and oleth-2 were further evaluated by varying the concentration in the formulation between 2.5-10% w/w. These two permeation enhancers were selected based on the initial rate and extent of active permeation through the membrane. In Examples 15-20, a bupivacaine topical spray formulation was prepared using the ingredients set forth in Table 7. Ethanol and povidone was used as solvent and polymer respectively.

The process for preparing the formulations is as follows:

Add drug, isopropyl myristate, polysorbate 80 and permeation enhancer in ethanol while stirring and mix till clear solution is formed. Add purified water drop by drop in drug:oil base solution with stirring until a nano-emulsion is formed. Add the film-forming polymer in the nano-emulsion while stirring till polymer dissolves or hydrates completely. Store the polymeric nano-emulsion in glass bottle with cap or mechanical pump.

The films were tested in In-vitro Franz Cell Strat-M synthetic membrane experiments. The results are set forth in Table 8 below:

TABLE 8
In-vitro cumulative drug permeation (ug/cm²)

| Time (Hr) | Ex 15 | Ex 16 | Ex 17 | Ex 18 | Ex 19 | Ex 20 |
|---|---|---|---|---|---|---|
| 0.5 | 2.13 | 3.70 | 4.97 | 1.76 | 3.08 | 2.76 |
| 1 | 12.55 | 33.42 | 41.34 | 11.74 | 39.21 | 26.32 |
| 2 | 95.08 | 220.63 | 234.56 | 182.63 | 302.15 | 216.83 |
| 4 | 459.51 | 635.85 | 599.48 | 648.62 | 649.14 | 633.11 |
| 6 | 679.53 | 769.52 | 725.15 | 735.48 | 706.03 | 704.43 |
| 24 | 1091.65 | 1152.72 | 1075.55 | 813.91 | 820.19 | 833.39 |

From the data, it was concluded that permeation enhancer concentration significantly improves the initial rate of permeation. In these Examples, the permeation enhancer concentration impacts Oleth-2 more than oleyl alcohol. Also, it was concluded that for Oleth-2, between 5-10 w/w concentration the impact of initial permeation rate was not that significant. It was decided that 7.5% w/w concentration of oleyl alcohol is an excellent permeation enhancer for the formulation.

### Examples 21-27

In Examples 21-27, a bupivacaine topical spray formulation was prepared using the ingredients set forth in Tables 9 and 10.

TABLE 9

| | Example 21 | Example 22 | Example 23 |
|---|---|---|---|
| | mg/spray | mg/spray | mg/spray |
| Bupivacaine | 8.00 | 8.00 | 8.00 |
| Povidone K30 | 6.00 | 6.00 | 6.00 |
| Propylene Glycol | 6.00 | 6.00 | 6.00 |
| Ethanol 95% | 90.94 | 90.94 | 90.94 |
| Menthol | 0.06 | 0.06 | 0.06 |
| Oleth - 2 | | 9.00 | |
| Oleic Acid | | | 9.00 |
| Isostearic Acid | 9.00 | | |
| Total | 120.00 | 120.00 | 120.00 |

TABLE 10

| | Example 24 | Example 25 | Example 26 | Example 27 |
|---|---|---|---|---|
| | mg/spray | mg/spray | mg/spray | mg/spray |
| Bupivacaine | | 8.00 | 8.00 | 4.00 |
| Base | | | | |
| Bupivacaine | 8.00 | | | |
| HCl | | | | |
| Povidone K30 | 6.00 | 6.00 | | 6.00 |
| Copovidone | | | 6.00 | |
| Propylene | 6.00 | 6.00 | 6.00 | 6.00 |
| Glycol | | | | |
| Ethanol 95% | 90.94 | 90.94 | 90.94 | 94.94 |
| Menthol | 0.06 | 0.06 | 0.06 | 0.06 |
| Oleyl Alcohol | 9.00 | 9.00 | | 9.00 |
| Isopropyl | | | 9.00 | |
| myristate | | | | |
| | 120.00 | 120.00 | 120.00 | 120.00 |

The process for preparing the formulations is as follows:

Add drug, propylene glycol, permeation enhancer and menthol in ethanol while stirring and mix till a clear solution is formed. Add the film-forming polymer in the clear drug solution while stirring till polymer dissolves or hydrates completely. Store the polymeric in glass bottle with cap or mechanical pump.

In-vitro permeation studies were performed on the formulations of Examples 21-27 on Mattek Epiderm and or cadaver skin using an In-line PermeGear ILC07 automatic diffusion cell system. While designing in-vitro skin permeation studies using human cadaver skin, the donor age group was considered in order to represent the target patient population. For development studies, human cadaver skin from a white male donor between age group of 50-60 years was selected. Human cadaver skin was received from the New York Firefighter Skin Bank as a cryopreserved split thickness skin allograft. A skin graft contains the epidermis and partial dermis that simulates the in-vivo diffusion barrier layer. The human cadaver skin was stored as received at temperature of −30° C. Before performing the in-vitro skin permeation studies the skin was submerged in the phosphate buffer solution at room temperature for 15 minutes. The in-vitro skin permeation studies were performed using a PermeGear ILC07 automatic diffusion cell system. Cumulative (μg/cm²) drug permeated through synthetic membrane with respect to time is provided in Table 11 below.

TABLE 11

| | Cumulative Drug Permeation ug/cm² | | | | | | |
|---|---|---|---|---|---|---|---|
| Hr | E-21 | E-22 | E-23 | E-24 | E-25 | E-26 | E-27 |
| 0 | 0 | 0 | 0 | 0 | 0 | 0 | 0 |
| 0.5 | 5 | 5 | 5 | 5 | 8 | 16 | 7 |
| 1 | 9 | 11 | 12 | 12 | 25 | 36 | 16 |
| 2 | 28 | 50 | 56 | 32 | 91 | 103 | 64 |
| 4 | 175 | 257 | 262 | 179 | 266 | 278 | 281 |
| 6 | 313 | 391 | 402 | 337 | 424 | 433 | 420 |
| 24 | 1020 | 955 | 1063 | 971 | 1792 | 1399 | 786 |

The cumulative (μg/cm²) drug permeated through human cadaver skin with respect to time is provided in Table 12 below with respect to Examples 24 and 25. Three different human cadaver skin donors were used to understand the impact of skin type on permeation rate. Seven diffusion cells were used per formulation per donor and the average of 21 permeation data points per formulation is reported in Table 12 below.

TABLE 12

Cumulative Drug Permeation ug/cm²

| Time | Example 24 | | | | Example 25 | | | |
|---|---|---|---|---|---|---|---|---|
| (Hr) | Donor 1 | Donor 2 | Donor 3 | Average | Donor 1 | Donor 2 | Donor 3 | Average |
| 0 | 0 | 0 | 0 | 0 | 0 | 0 | 0 | 0 |
| 0.5 | 10 | 16 | 25 | 17 | 22 | 14 | 4 | 13 |
| 1 | 18 | 52 | 43 | 38 | 38 | 34 | 8 | 27 |
| 2 | 36 | 177 | 86 | 100 | 86 | 90 | 19 | 65 |
| 4 | 223 | 651 | 289 | 387 | 244 | 234 | 59 | 179 |
| 6 | 489 | 963 | 472 | 641 | 396 | 373 | 110 | 293 |
| 24 | 1787 | 2080 | 1056 | 1641 | 1356 | 1633 | 639 | 1209 |

Based on the above data, it was concluded that bupivacaine hydrochloride (Example 24) demonstrates superior permeation than bupivacaine base (Example 25) in the three different skin donors.

### Example 28

In Example 28, particle size distribution data for the bupivacaine metered dose transdermal film spray of Example 24 is provided in Table 13 below:

TABLE 13
Bupivacaine topical spray droplet size distribution data

| | Unit ID | | | | |
|---|---|---|---|---|---|
| | | | | | Pump |
| | Dv(10), | Dv(50), | Dv(90), | % Droplets < | Delivery |
| | μm | μm | μm | 10 μm | (mg) |
| Example 24 - | 27.63 | 56.77 | 121.9 | 1.497 | 126.1 |
| first spray | | | | | |
| attempt | | | | | |
| Example 24 - | 26.01 | 54.04 | 114.9 | 1.593 | 125.8 |
| second spray | | | | | |
| attempt | | | | | |
| Example 24 - | 24 | 52.01 | 112.4 | 1.740 | 125.0 |
| third spray | | | | | |
| attempt | | | | | |
| Mean | 25.88 | 54.27 | 116.4 | 1.610 | 125.6 |
| SD | 1.82 | 2.39 | 4.9 | 0.12 | 0.6 |
| % CV | 7.0 | 4.4 | 4.2 | 7.6 | 0.5 |

### Example 29: In-Vivo Study

An in-vivo study was performed in healthy rats to evaluate drug release from a bupivacaine topical bio-adhesive film forming spray made in accordance with Example 24. A total of 4 sprays were applied (two sprays on the back and two sprays on the abdominal area of the animal) using a metered dose mechanical spray pump. Blood samples were collected at 15, 30 minutes, 1, 2, 4, 8 and 24 hours. All samples were analyzed using a validated analytical LC-MS method.

The permeation or release of the drug following topical application from the invention is characterized by a biphasic release profile, the first phase comprising an immediate release of drug from the drug-solvent micro-droplet that is facilitated by the small (approximately 50 microns) spray droplet size and a subsequent second controlled release phase comprising release of drug from the bio-adhesive film formed, upon evaporation of the solvent system.

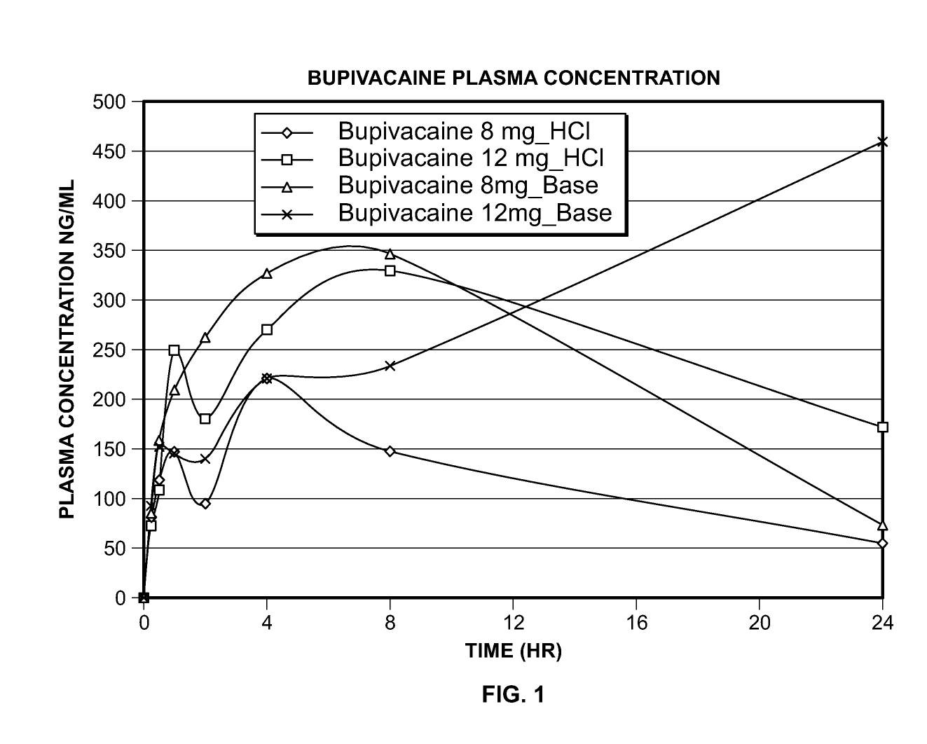

The results obtained are reported in Table 14 and FIGS. 1-4. It can be concluded from the results that the topical/transdermal drug delivery formulations of the invention preferably have a bi-phasic release profile and the drug release is dose proportional. Both the 8 and 12 mg strengths show the first phase of drug release at about 1 hour after spray application, signifying that spray pattern and droplet size is very critical in achieving immediate drug permeation and fast onset of action. The second slower phase of drug release observed between 4-8 hours after spray application indicates that drug gradually permeates from the polymeric bio-adhesive film in a controlled manner. This observed in-vivo bi-phasic drug release through the topical/transdermal route is unique to this invention—a drug containing polymeric bio-adhesive film spray. It is also clear from the data that the lag in Cmax during the second phase of drug release at higher drug concentrations signifies a prolongation in effect as dosage strength is increased.

TABLE 14
Plasma concentration of drug at various time-points

| | Average drug plasma concentration ng/ml | | |
|---|---|---|---|
| | Bupivacaine HCl | Bupivacaine HCl | |
| Time (Hr) | 8 mg | 12 mg | Placebo |
| 0 | 1 | 0 | 0 |
| 0.25 | 82 | 73 | 23 |
| 0.5 | 119 | 109 | 35 |
| 1 | 148 | 249 | 50 |
| 2 | 95 | 180 | 48 |
| 4 | 221 | 270 | 39 |
| 8 | 147 | 329 | 49 |
| 24 | 55 | 172 | 8 |

FIG. 1 depicts an in-vivo drug plasma concentration vs time plot of individual rats treated with a bupivacaine metered dose transdermal film spray in accordance with the present invention, bupivacaine HCl 8 mg (n=5), bupivacaine HCl 12 mg (n=3), bupivacaine base 8 mg (n=7), and bupivacaine base 12 mg (n=3).

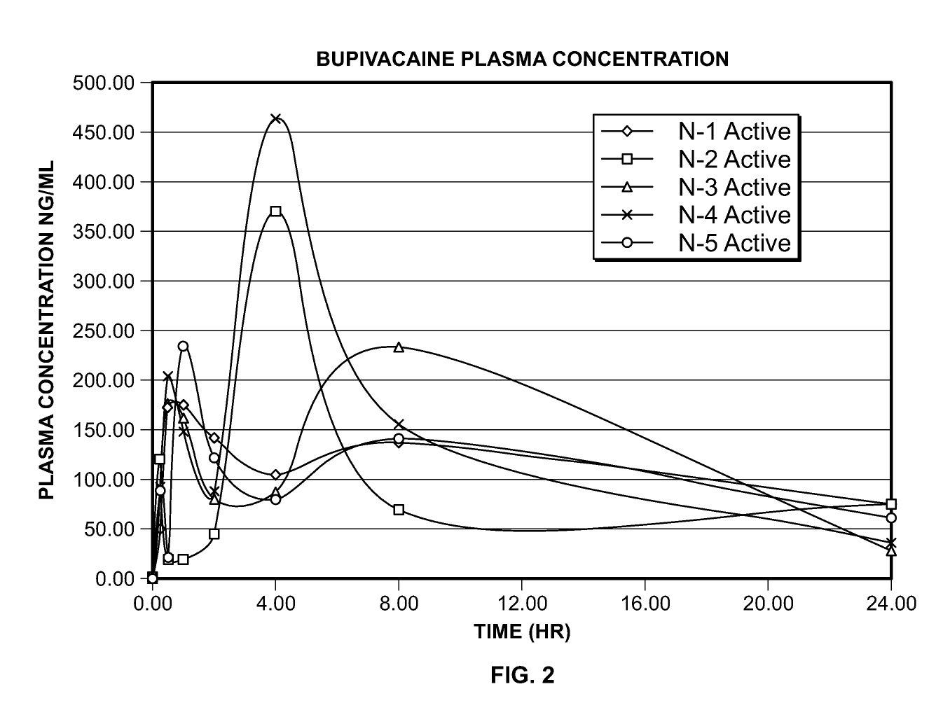

FIG. 2 depicts an in-vivo drug plasma concentration vs time plot of individual rats treated with a bupivacaine metered dose transdermal film spray in accordance with the present invention.

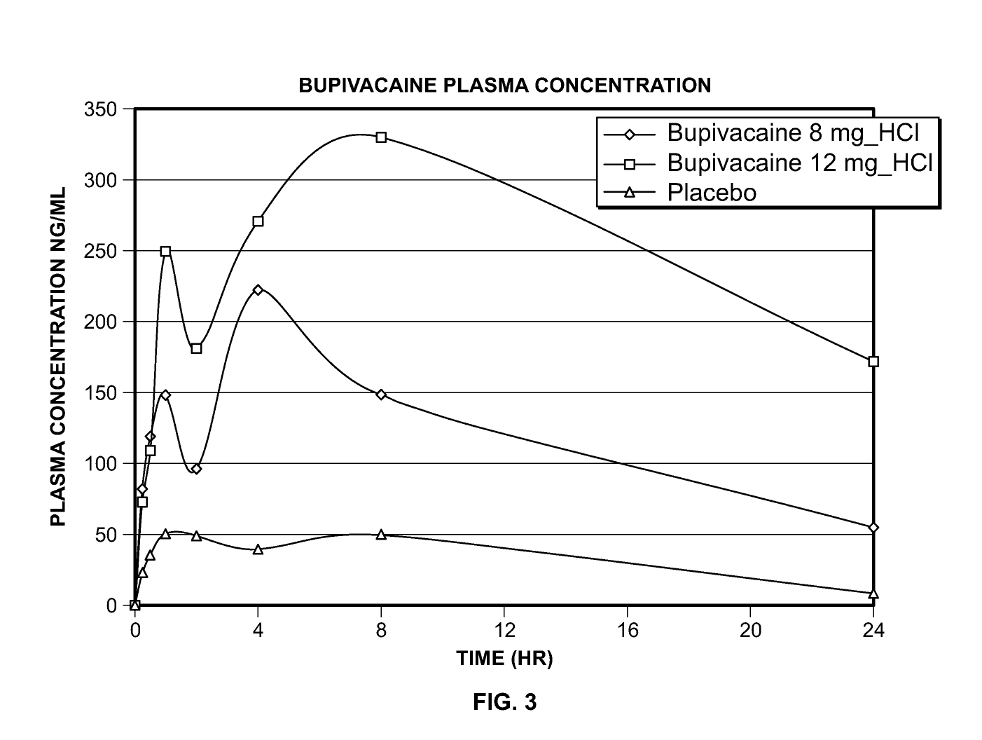

FIG. 3 depicts an in-vivo average drug plasma concentration vs time plot of 8 mg, 12 mg and placebo of individual rats treated with a bupivacaine metered dose transdermal film spray in accordance with the present invention.

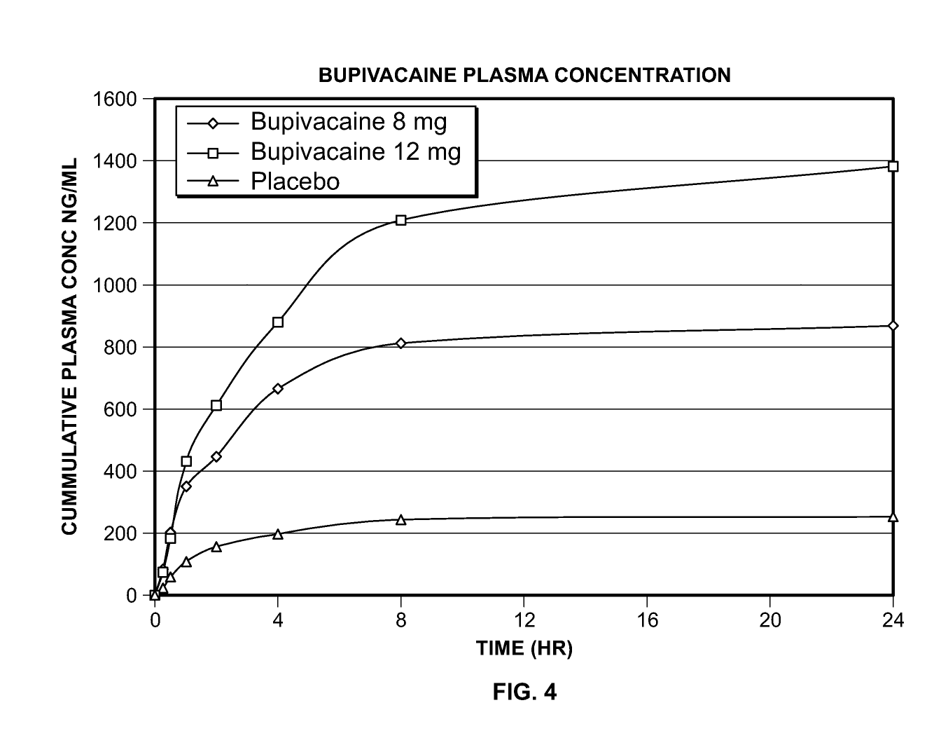

FIG. 4 depicts an in-vivo average cumulative drug plasma concentration vs time plot of 8 mg, 12 mg and placebo of individual rats treated with a bupivacaine metered dose transdermal film spray in accordance with the present invention.

### Examples 30-32

In Examples 30-32, local anesthetic topical spray formulations were prepared using the ingredients set forth in Table 15 for bupivacaine base, ropivacaine base and lidocaine HCl.

The process for preparing the formulations is as follows:

Add drug, propylene glycol, permeation enhancer and menthol in ethanol while stirring and mix till a clear solution is formed. Add the film-forming polymer in the clear drug solution while stirring till polymer dissolves or hydrates completely. Store the polymeric in glass bottle with cap or mechanical pump.

The films were tested in In-vitro Franz Cell Strat-M synthetic membrane experiments. The results are set forth in Table 16 below:

TABLE 16
In-vitro cumulative drug permeation (ug/cm²)

| | Ex 30 - (Bupi | Ex 31- (Ropi | Ex 32 - (Lido |
|---|---|---|---|
| Time (Hr) | Base) | Base) | HCl) |
| 0.5 | 1.78 | 0.56 | 2.25 |
| 1 | 8.04 | 10.81 | 41.75 |
| 2 | 41.97 | 76.95 | 283.76 |
| 4 | 172.65 | 314.58 | 519.55 |
| 6 | 272.59 | 463.83 | 569.71 |
| 24 | 566.76 | 744.90 | 637.97 |

### Example 33

In Examples 33a-c, a testosterone topical spray formulation was prepared using the ingredients set forth in Table 17.

The method of preparation is similar to the procedure set forth above for Examples 2-5.

### Example 34

In Examples 34a-c, a ketamine topical spray formulation was prepared using the ingredients set forth in Table 18.

The method of preparation is similar to the procedure set forth above for Examples 2-5.

### Example 35

In Examples 35a-b, an estradiol topical spray formulation was prepared using the ingredients set forth in Table 19.

The method of preparation is similar to the procedure set forth above for Examples 2-5.

### Example 36

In Example 36, a fentanyl topical spray formulation was prepared using the ingredients set forth in Table 20.

TABLE 20

| | mg/spray |
|---|---|
| Fentanyl | 0.1-2.0 |
| Povidone K30 | 6.00 |
| Propylene Glycol | 6.00 |
| Ethanol 95% | Qs |
| Menthol | 0.06 |
| Oleyl Alcohol | 9.00 |

The method of preparation is similar to the procedure set forth above for Examples 2-5.

### Example 37

In Examples 37a-b, a methylphenidate topical spray formulation was prepared using the ingredients set forth in Table 21.

The method of preparation is similar to the procedure set forth above for Examples 2-5.

### Example 38

In Examples 38a-b, a neomycin topical spray formulation was prepared using the ingredients set forth in Table 22.

The method of preparation is similar to the procedure set forth above for Examples 2-5.

### Example 39

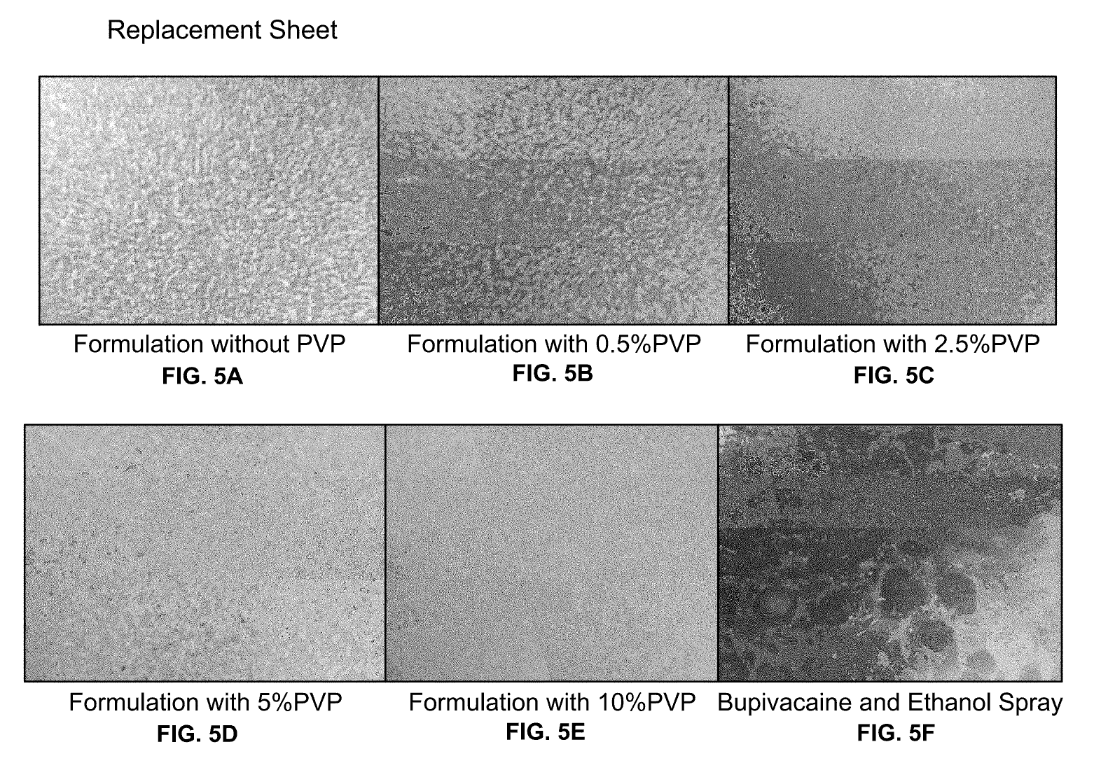

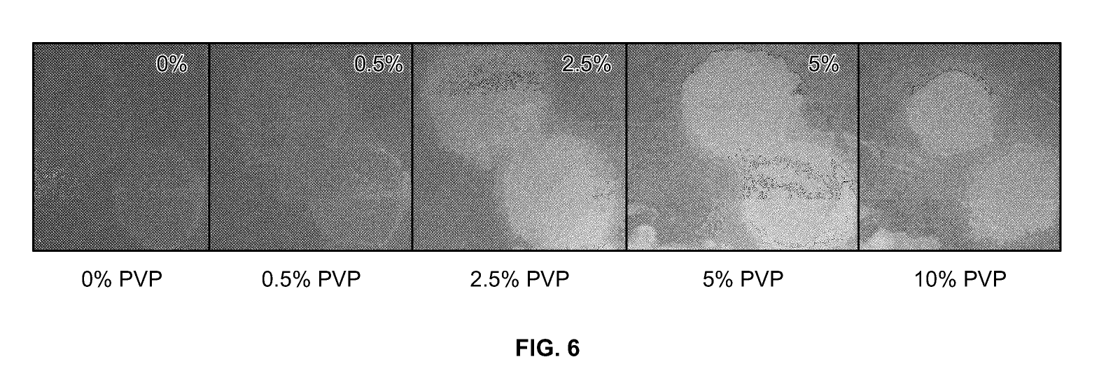

Bupivacaine topical spray formulations were prepared using different concentrations of polyvinyl pyrrolidone (PVP) (0.0%, 0.5%, 2.5%, 5% and 10%) and sprayed over glass slides to evaluate the rate and extent of bupivacaine crystal precipitation after topical spray film formation. FIGS. 5 and 6 visually illustrate the bupivacaine topical spray film characteristics. More specifically, FIG. 5 depicts bupivacaine topical spray film characteristics in formulations according to the present invention a) without PVP (significant bupivacaine crystals observed); b) formulation with 0.5% PVP (significant bupivacaine crystals observed; c) formulation with 2.5% PVP; d) formulation with 5% PVP; e) formulation with 10% PVP; and f) bupivacaine and ethanol spray (significant bupivacaine crystals observed). It was observed that the rate and extent of bupivacaine crystal precipitation significantly decrease with increase in the formulation polymer concentration. It was also observed that the uniformity and integrity of film increases with the increase in polymer concentration. More specifically, FIG. 6 depicts the spray pattern and film uniformity of bupivacaine spray formulations according to the present invention containing different concentrations of the precipitation inhibiting polymer PVP (0% PVP, 0.5% PVP; 2.5% PVP; 5% PVP; and 10% PVP. It was concluded that a PVP concentration in the formulation of between 2.5% and 10% significantly inhibits bupivacaine precipitation and forms a uniform film after solvent evaporation.

### Example 40

In Examples 40a-b, a bupivacaine HCl topical spray formulation was prepared using the ingredients set forth in Table 23.

The process for preparing the formulations is as follows:

Add drug, propylene glycol, permeation enhancer, ascorbic acid (antioxidant agent) and menthol in ethanol while stirring and mix till a clear solution is formed. Add the film-forming polymer in the clear drug solution while stirring till polymer dissolves or hydrates completely. Store the polymeric in glass bottle with cap or mechanical pump.

### Example 41

In Example 41, a single dose crossover study to evaluate the pharmacokinetics and relative bioavailability of 3 doses of bupivacaine topical spray and bupivacaine injectable in healthy male and female volunteers was conducted. The pharmacokinetic study was performed in 12 healthy human volunteers to assess the safety and tolerability of bupivacaine topical spray delivery system and estimate the duration time of local effect perceived by the healthy volunteers following single escalating doses of bupivacaine topical spray. The reference therapy was a single 30 mg dose (a total of 6 ml, 3 injections of 2 ml) of bupivacaine HCl administered subcutaneously.

The test formulation was a bupivacaine hydrochloride 10 mg topical spray (corresponding to 8.88 mg of bupivacaine base) prepared in accordance with Example 40b. The reference formulation was Sensorcaine® (bupivacaine hydrochloride) 0.5% 10 mL.

A single subcutaneous dose of Treatment-A or a single topical dose of Treatment-B, -C, or -D was administered according to the following randomization scheme: Treatment-A: Single dose of 30 mg: 3 subcutaneous injections (2 mL each) of Sensorcaine® (Bupivacaine Hydrochloride) 0.5% 10 mL (Reference); Treatment-B: Single dose of 30 mg: 3 sprays of GTX101 (Bupivacaine Hydrochloride 10 mg Topical Spray; Test); Treatment-C: Single dose of 50 mg: 5 sprays of GTX101 (Bupivacaine Hydrochloride 10 mg Topical Spray; Test); Treatment-D: Single dose of 70 mg: 7 sprays of GTX101 (Bupivacaine Hydrochloride 10 mg Topical Spray; Test).

Two treatments (Treatment-A and either Treatment-B, -C or -D) were to be administered to 12 subjects as described in Table 24. Subjects arrived at the clinical site at least 10 hours before dosing. After a supervised overnight fast, subjects received a standard breakfast before drug administration following which, a single subcutaneous dose of bupivacaine hydrochloride 30 mg or a single topical dose of bupivacaine hydrochloride 30 mg, 50 mg or 70 mg was administered in the morning. Subjects were allowed to leave the clinical site after the 24-hour post dose blood draw. The wash-out period was at least 3 calendar days.

TABLE 24
Study Sequences

| | Period 1 | Period 2 |
|---|---|---|
| Sequence 1 (n = 2) | Treatment-A | Treatment-B |
| Sequence 2 (n = 2) | Treatment-B | Treatment-A |
| Sequence 3 (n = 2) | Treatment-A | Treatment-C |
| Sequence 4 (n = 2) | Treatment-C | Treatment-A |
| Sequence 5 (n = 2) | Treatment-A | Treatment-D |
| Sequence 6 (n = 2) | Treatment-D | Treatment-A |

All 12 volunteers completed the study. In each study period, 21 blood samples were collected. The first sample was collected prior to drug administration while the others were collected up to 24 hours after the drug administration. Additionally, skin irritation was assessed at the application site approximately 5 minutes, 6 hours, and 24 hours after dosing for each treatment. At each time point the local skin reaction was assessed and recorded on a specific form included in the Case Report Form. All 12 subjects showed no changes in their skin irritation assessments, all dermal responses were scored as 0; there was no evidence of irritation and no other effects were observed.

### Perception Analysis (Efficacy Analysis)

Subjects were questioned if they felt the sensation of a Q-Tip (Yes/No) at every 30 minutes after drug application for the first 8 hours or until sensation returned whichever came first. Two Q-Tip tests were performed one at the site of application and another at a location away from the site of application. The Q-Tip test was performed by lightly touching the Q-Tip on the surface of skin. All local effects perceived by the subjects that persisted 8 hours after drug administration were reported as adverse events. Both the reference and test products shows similar results for the loss in sensation after Q-tip analysis which suggest that the novel bupivacaine topical spray is equally efficacious as reference subcutaneous injectable product. The Q-tip perception analysis results are reported in Table 25.

TABLE 25
Summary of Q-Tip perception analysis

| | Reported loss of Q-tip sensation (Yes/No) | |
|---|---|---|
| | in the first 8 hours after dosing | |
| Subject | Topical Spray | SC Injection |
| 1 | Yes | No |
| 2 | No | Yes |
| 3 | No | Yes |
| 4 | Yes | Yes |
| 5 | Yes | No |
| 6 | Yes | No |
| 7 | Yes | Yes |
| 8 | No | No |
| 9 | No | Yes |
| 10 | Yes | Yes |
| 11 | Yes | No |
| 12 | No | Yes |
| Total # of subjects reported | 7 out of 12 | 7 out of 12 |
| loss in Q-tip sensation | | |
| % Subject reported loss in | 58.3% | 58.3% |
| Q-tip sensation | | |

The pharmacokinetic results are reported in Table 26. Tmax is shown in units of hours and Cmax is in units of ng/mL.

TABLE 26
Summary of plasma bupivacaine pharmacokinetic parameters

| | Treatment A (n = 12) | | Treatment B (n = 4) | | Treatment C (n = 4) | | Treatment D (n = 4) | |
|---|---|---|---|---|---|---|---|---|
| Parameter | 30 mg SC | | 30 mg Spray | | 50 mg Spray | | 70 mg Spray | |
| (Units) | Mean | (C.V. %) | Mean | (C.V. %) | Mean | (C.V. %) | Mean | (C.V. %) |
| Cₘₐₓ (ng/mL) | 129.31 | (41.5) | 1.03 | NC | 2.41 | (44.6) | 1.55 | (31.3) |
| Tₘₐₓ (hours) | 0.33 | (0.17-0.67) | 24.00 | NC | 24.00 | (24.00-24.05) | 24.00 | (2.00-24.00) |

NC Not calculated

From a single dose application over 24 hours, a mean maximum plasma concentration of 2.41 ng/mL for topical spray formulation and 129.31 ng/mL for reference injectable product was observed. From the above clinical study, it can be concluded that the novel bupivacaine topical spray formulation is effective, safe and well tolerated.

### Example 42

In Example 42, an open-label study to evaluate the single dose pharmacokinetics of bupivacaine topical spray in healthy male and female volunteers was conducted. The pharmacokinetic study was performed in 10 healthy human volunteers to assess the safety, tolerability of a 100 mg dose of topical bupivacaine administered as 10 topical sprays. The study was a single center, non-randomized, single dose, open-label, one period, one treatment design. A 100 mg dose of bupivacaine was administered topically (10 sprays applied in a similar distribution covering the skin surface at the T10 dermatome). The bupivacaine spray provided about 10 mg bupivacaine per spray. The formulation was prepared in accordance with Example 40b.

All 10 volunteers completed the study. 29 blood samples were collected per subject. The first sample was collected prior to drug administration while the others were collected up to 504 hours after drug administration. Additionally, skin irritation was assessed at the application site approximately 5 minutes, 6 hours, and 24 hours after dosing for each treatment. At each time point the local skin reaction was assessed and recorded on a specific form included in the Case Report Form. All 10 subjects showed no changes in their skin irritation assessments, all dermal responses were scored as 0; there was no evidence of irritation and no other effects were observed.

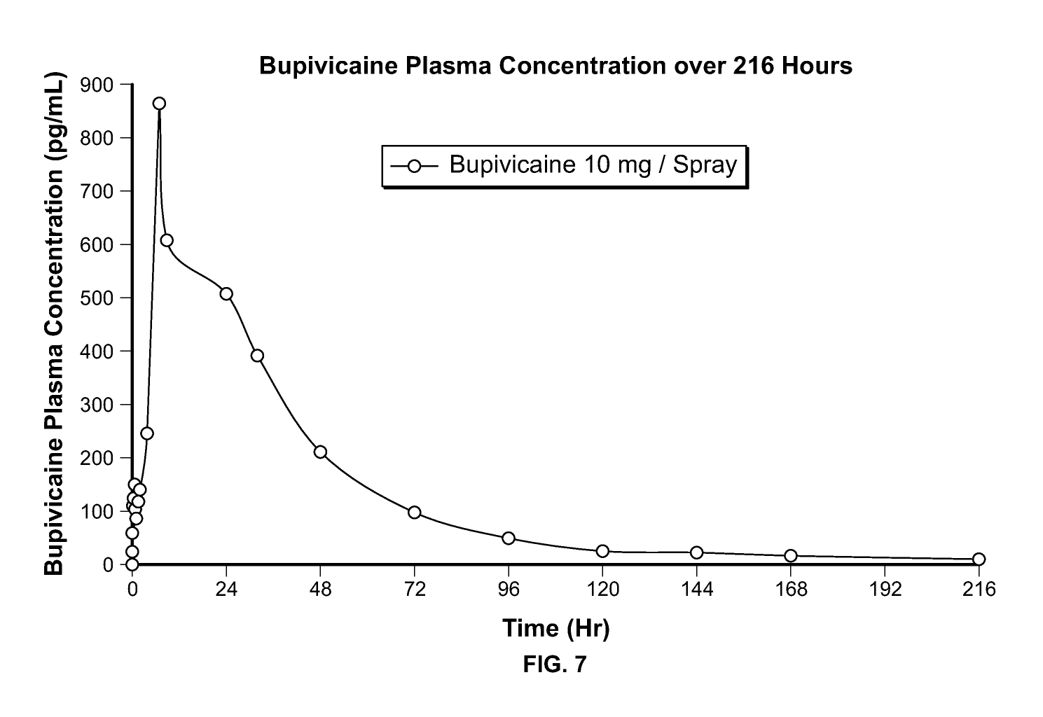

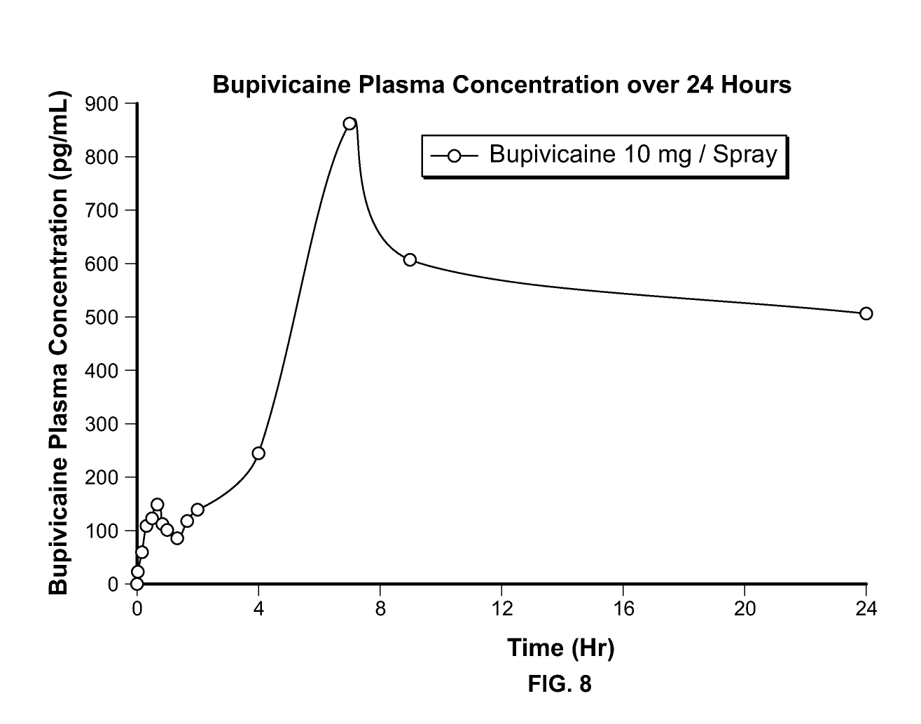

The pharmacokinetic results are reported in FIGS. 7 and 8 and Table 26. Tmax is shown in units of hours and Cmax is in units of pg/mL. FIG. 7 is a graph depicting the bupivacaine plasma concentration (pg/mL) over 216 hours. FIG. 8 is a graph depicting the bupivacaine plasma concentration over 24 hours.

TABLE 26
Summary of plasma bupivacaine pharmacokinetic parameters

| Parameter (Units) | Mean | (C.V. %) |
|---|---|---|
| Cₘₐₓ (pg/mL) | 1248.72 | 92.1 |
| Tₘₐₓ (hours)^{a} | 11.97 | (0.67-24.00) |
| AUC_{0-T} (pg · h/mL) | 29493.41 | 66.5 |
| T_{half} (hours) | 33.86 | −75.6 |

From a single dose application over 504 hours, a mean maximum plasma concentration of 1248 pg/mL was observed. It can be concluded from FIGS. 7 and 8 that this topical/transdermal drug delivery technology invention has a bi-phasic release profile similar to that observed in the rat study. The first phase of drug releases at about 30-40 minutes after spray application, signifying that spray pattern and droplet size are critical to achieving immediate drug permeation and fast onset of action. The second slower phase of drug release observed between 7-24 hours after spray application indicates that drug gradually permeates from the polymeric bio-adhesive film in a controlled manner. This observed in-vivo bi-phasic drug release through the topical/transdermal route is unique to the present invention—a drug-containing polymeric bio-adhesive topical film spray. It is also clear from the data that the lag in Cmax during the second phase of drug release at higher drug concentrations signifies a prolongation in effect as dosage strength is increased. Table 27 provides blood sample times and plasma concentration breakdowns for both peaks in the individual subjects.

### Examples 43a-b

In-vitro permeation studies were conducted on formulations prepared in accordance with the invention.

In Example 43a, the formulation of Example 12 was tested for cumulative drug permeation per cm²cm2 from a Strat-M synthetic membrane. Over the first two hours, the drug permeation ranged from 10 μg/cm² to 500 μg/cm². Over 24 hours, the drug permeation ranged from 10 μg/cm² to 6500 μg/cm².

In Example 43b, the formulation of Example 24 was tested for cumulative drug permeation per cm2 from Mattek and human cadaver skin membrane. Over the first two hours, the drug permeation ranged from 10 μg/cm² to 500 μg/cm². Over 24 hours, the drug permeation ranged from 10 μg/cm² to 6500 μg/cm².

### Examples 44a-b

In-vivo permeation studies were conducted on formulations prepared in accordance with the invention.

In Example 44a, the formulation of Example 24 was tested for cumulative drug permeation per cm² on rat skin. Over the first two hours, the drug permeation ranged from 10 ng/cm² to 500 ng/cm². Over 24 hours, the drug permeation ranged from 10 ng/cm² to 6500 ng/cm².

In Example 44b, the formulation of Example 40b was tested for cumulative drug permeation per cm² on human skin. Over the first two hours, the drug permeation ranged from 10 ng/cm² to 500 ng/cm². Over 24 hours, the drug permeation ranged from 10 ng/cm² to 6500 ng/cm².

### Example 45

Solubility studies to find out concentration at which the drug is present at saturation levels were performed as follows: The drug (bupivacaine hydrochloride 2, 4, 8, 10, 12 & 16 mg) was added to, propylene glycol, oleyl alcohol and menthol in ethanol under stirring and mixing till clear solution is formed. The film-forming polymer in the clear drug solution was added while stirring till polymer dissolves or hydrates completely. In the case of 16 mg of drug solution the drug precipitated out from clear solution after standing. Other solutions were clear and no precipitate was observed. It can be concluded that drug saturation was achieved at a concentration of about 12 mg per spray.

### CONCLUSION

It will be readily apparent to one of ordinary skill in the relevant arts that other suitable modifications and adaptations to the methods and applications described herein are suitable and may be made without departing from the scope of the invention or any embodiment thereof. While the invention has been described in connection with certain embodiments, it is not intended to limit the invention to the particular forms set forth, but on the contrary, it is intended to cover such alternatives, modifications and equivalents as may be included within the spirit and scope of the invention as defined by the following claims.

1. A method of treating pain, comprising spraying onto the skin of a human a unit dose of a topical spray pharmaceutical formulation comprising a polymeric solution, emulsion or suspension of a hydrophilic polymer, a drug crystal precipitation inhibiting agent, an effective amount of an active agent suitable for treating pain, and a pharmaceutically acceptable permeation enhancer dispersed in a pharmaceutically acceptable hydroalcoholic solvent, the unit dose comprising a plurality of spray droplets, wherein 10% of the spray droplets in the unit dose have a mean diameter of about 26 μm±20 μm, about 50% of the spray droplets in the unit dose have a mean diameter of about 55 μm±20 μm, and about 90% of the spray droplets in the unit dose have a mean diameter of about 116 μm±40 μm, such that the unit dose topical spray provides a film surface area from about 1 cm² to about 40 cm² per spray and sets as a microporous, breathable and bioadhesive film when the hydroalcoholic solvent evaporates and provides a biphasic release of the active agent.

2. The method of claim 1, wherein the active agent is selected from the group consisting of bupivacaine base, bupivacaine hydrochloride, and a combination thereof in an amount from about 0.5 to about 40 mg, based on bupivacaine hydrochloride.

3. The method of claim 1, wherein the active agent is selected from the group consisting of an anesthetic, steroid, an opioid, a non-steroidal anti-inflammatory agent (NSAID), a central nervous system stimulant, an anti-bacterial, and combinations of any of the foregoing.

4. The method of claim 1, which provides (i) an in-vitro cumulative drug permeation from about 10 ug/cm² to about 500 ug/cm² after 2 hours, and a cumulative drug permeation from 10 μg/cm² to 6500 μg/cm² after 24 hours and/or (ii) an in-vivo cumulative drug permeation on human skin from about 10 ng/cm² to about 500 ng/cm² after 2 hours, and a cumulative drug permeation from about 10 ng/cm²cm2 to about 6500 ng/cm² after 24 hours.

5. The method of claim 1, wherein the biphasic release provides a first peak concentration of the active agent at from about 0.5 to about 3 hours and the second peak concentration of the active agent at from about 3 to about 15 hours after application of the unit dose on human skin.

6. The method of claim 1, wherein the biphasic release provides a first peak at from about 0.5 to about 3 hours and a second peak at from about 3 to about 7 hours after application of the unit dose on human skin.

7. The method of claim 2, wherein the hydrophilic polymer comprises from about 2 to about 50% of the formulation, and the drug crystal precipitation inhibiting agent comprises from about 2.5 to about 10% of the formulation, by weight and wherein the bupivacaine concentration in the formulation is supersaturated.

8. The method of claim 7, wherein the supersaturated drug concentration lasts for a time period from about 1 to about 24 hours in vivo to achieve increased bioavailability.

9. The method of claim 1 wherein the human is treated for neuropathic pain.

10. The method of claim 1, wherein the human has a disease selected from the group consisting of general pain, neuropathic pain neuropathic pain, postoperative pain, sports pain, osteoporosis pain, pain resulting from cosmetic procedures, dental pain, wound pain and burn pain.

11. The method of claim 1, wherein the human is treated for pain arising from post-herpetic neuralgia.

12. A method of treating pain, comprising spraying onto the skin of a human in proximity to an affected area a unit dose of a topical spray pharmaceutical formulation comprising a polymeric solution, emulsion or suspension of a hydrophilic polymer, a drug crystal precipitation inhibiting agent, an effective amount of an active agent comprising a local anesthetic or a pharmaceutically acceptable salt thereof, and a pharmaceutically acceptable permeation enhancer dispersed in a pharmaceutically acceptable hydroalcoholic solvent, the unit dose comprising a plurality of spray droplets, wherein 10% of the spray droplets in the unit dose have a mean diameter of about 26 μm±20 μm, about 50% of the spray droplets in the unit dose have a mean diameter of about 55 μm±20 μm, and about 90% of the spray droplets in the unit dose have a mean diameter of about 116 μm±40 μm, such that the unit dose topical spray sets as a microporous, breathable and bioadhesive film having a film surface area from about 1 cm² to about 40 cm² per spray and when the hydroalcoholic solvent evaporates and provides a biphasic release of the active agent.

13. The method of claim 12, further comprising using a metered pump delivering, e.g., from about 40 μl to about 350 μl volume per spray to deliver the active agent onto the skin of the human.

14. The method of claim 12, wherein the unit dose comprises from about 1 to about 20 sprays of the topical spray pharmaceutical formulation.

15. The method of claim 13, wherein the local anesthetic is bupivacaine hydrochloride in an amount from about 0.5 to about 40 mg.

16. The method of claim 15, wherein the biphasic release provides a first and second phase of bupivacaine release, wherein the first phase of bupivacaine release reaches a peak at from about 0.25 to about 1.5 hours and the second phase of bupivacaine release reaches a peak plasma concentration at from about 4 to about 12 hours after application of the unit dose on human skin.

17. The method of claim 15, wherein the bupivacaine is supersaturated in the topical spray formulation.

18. The method of claim 12, further comprising topically spraying additional unit doses of the topical spray pharmaceutical formulation as needed to treat pain in the human.

19. The method of claim 12, wherein the human is suffering from neuropathic pain.

20. The method of claim 18, wherein the neuropathic pain is selected from the group consisting of erythromelalgia, post-herpetic neuralgia (PHN), fibromyalgia and complex regional pain syndrome (CRPS).

21. The method of claim 16, wherein 10% of the spray droplets in the unit dose have a mean diameter of about 26 μm±1.82 μm, about 50% of the spray droplets in the unit dose have a mean diameter of about 55 μm±2.39 μm, and about 90% of the spray droplets in the unit dose have a mean diameter of about 116 μm±4.9 μm.

22. The method of claim 15, wherein the biphasic release provides a first peak concentration which occurs at from about 0.17 to about 0.67 hours after the unit dose is sprayed onto the human subject, and provides a second peak concentration which occurs at from about 4 to about 24 hours after the unit dose is sprayed onto the human subject.

23. The method of unit dose of claim 22, wherein the unit dose provides a first peak plasma concentration from about 29 pg/ml to about 380 pg/ml bupivacaine, and a second peak plasma concentration from about 864 pg/ml to about 3463 pg/ml bupivacaine.
